(19) United States
(12) Patent Application Publication
(10) Pub. No.: US 2026/0027048 A1
(43) Pub. Date: Jan. 29, 2026

(54) GEMFIBROZIL FORMULATION

(71) Applicant: POLARYX THERAPEUTICS, INC., Paramus, NJ (US)

(72) Inventor: Hahn-Jun LEE, Paramus, NJ (US)

(73) Assignee: POLARYX THERAPEUTICS, INC., Paramus, NJ (US)

(21) Appl. No.: 18/871,396

(22) PCT Filed: Jun. 2, 2023
(86) PCT No.: PCT/US2023/024244

§ 371 Date: Dec. 3, 2024

Related U.S. Application Data
(60) Provisional application No. 63/348,608, filed on Jun. 3, 2022.

(51) Int. Cl. A61K 9/08; A61K 9/00; A61K 31/192; A61K 45/06; A61K 47/02; A61K 47/18; A61K 47/22; A61K 47/26; A61K 47/46

(52) U.S. Cl. CPC A61K 9/08; A61K 9/0053; A61K 31/192; A61K 45/06; A61K 47/02; A61K 47/183; A61K 47/186; A61K 47/22; A61K 47/26; A61K 47/46

(57) ABSTRACT

In an aspect, the disclosure relates to a liquid pharmaceutical composition comprising, consisting essentially of, or consisting of gemfibrozil and an aqueous solvent. The disclosure also relates to methods of making the liquid pharmaceutical composition comprising gemfibrozil, uses thereof, and methods of treatment comprising administering the liquid pharmaceutical composition comprising gemfibrozil.

### CROSS REFERENCE TO RELATED APPLICATIONS

This application is a 35 USC § 371 National Stage application of International Patent Application No. PCT/US2023/024244, which was filed Jun. 2, 2023, entitled “GEMFIBROZIL FORMULATION” and claims priority to U.S. Provisional Patent Application No. 63/348,608, filed Jun. 3, 2022, which are both incorporated herein by reference as if fully set forth.

### FIELD OF INVENTION

The disclosure herein relates to an oral liquid pharmaceutical composition comprising gemfibrozil. The gemfibrozil in the composition is freely soluble in aqueous solutions and stable during storage. The disclosure also relates to methods for preparing such an oral liquid pharmaceutical composition, and methods of treatment comprising administering the oral liquid pharmaceutical composition.

### BACKGROUND

Gemfibrozil, or 5-(2,5-dimethylphenoxy)-2,2-dimethyl-pentanoic acid, is a fibrate compound, which is a class of drugs that have been shown to increase high density lipoproteins (HDL) and lower plasma triglyceride levels. Gemfibrozil may also affect lysosomal storage disorders including Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis, and Neuronal Ceroid Lipofuscinoses as well as neurodegenerative disorders including Alzheimer's disease, Parkinson's disease, Dementia with Lewy Bodies, Multiple System Atrophies, Multiple Sclerosis, Huntington's disease, and Amyotrophic Lateral Sclerosis. The mechanism by which gemfibrozil decreases these abnormal accumulations is not entirely clear, but may involve peroxisome proliferator activated receptor (PPAR) a mediated pathway. Gemfibrozil may provide overall enhanced lysosomal biogenesis resulting in decreased accumulations of lipofuscins within cells of the CNS in those affected by an NCL or other lysosomal storage disorder.

Gemfibrozil, is typically administered orally. But, its long hydrophobic alkyl-benzene backbone renders it poorly soluble in many aqueous solutions. The solubility of gemfibrozil in water is approximately 0.03 mg/ml compared with its solubility in organic buffers, such as ethanol and DMSO where solubilities are approximately 30 mg/ml and 16 mg/ml, respectively. Liquid formulations of gemfibrozil and other fibrate have been manufactured using a premix of one or more solvents and surfactants. Typical solvents used in liquid formulations of gemfibrozil include glycerol, propylene glycol, and polyethylene glycol. Surfactants used in such formulations include Tween 80, glyceryl monooleate, Cremephor ELP, Span 80, Poloxamer P188, Labrafac CM 10, Maisine 35-1, Solutol HS, vitamin E TPGS, and antioxidants such as butylated hydroxyanisole (BHA) and butylated hydroxytoluene (BHT).

An aqueous solvent for providing a liquid formulation for oral administration comprising gemfibrozil with good solubility, stability, microbial purity, and self-preserving ability would most likely lead to a liquid formulation with improved dissolution when taken orally, as well as increased bioavailability.

### SUMMARY

In an aspect, the invention relates to a liquid pharmaceutical gemfibrozil composition comprising, consisting essentially of, or consisting of gemfibrozil and an aqueous solvent. The gemfibrozil is soluble in the liquid pharmaceutical composition and has a concentration of approximately 5 mg/mL to 32 mg/ml. The solvent is an aqueous buffer having pH 10.5 (+/−0.5). The solvent may be a carbonate buffer having pH 10.5 (+/−0.5). The liquid pharmaceutical composition is stable for at least 36 months at storage temperatures in the range of 2° C. to 40° C.

In an aspect, the invention relates to a method for generating a liquid pharmaceutical composition comprising gemfibrozil, the method comprising, consisting essentially of, or consisting of adding gemfibrozil to an aqueous solvent and mixing at temperatures in the range of 40-65° C. until solids dissolve and the final concentration of gemfibrozil in the liquid pharmaceutical composition is 5 mg/ml to 32 mg/ml. The solvent may be an aqueous buffer having pH 10.5 (+/−0.5). The solvent may be a carbonate buffer having pH 10.5 (+/−0.5).

In an aspect, the invention relates to a method of treating a subject having lysosomal storage disorders, including Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis, Neuronal Ceroid Lipofuscinoses, and neurodegenerative disorders, including Alzheimer's disease, Parkinson's disease, Dementia with Lewy Bodies, Multiple System Atrophies, Multiple Sclerosis, Huntington's disease, and Amyotrophic Lateral Sclerosis. The method comprises administering a liquid pharmaceutical composition comprising gemfibrozil and an aqueous solvent, where the gemfibrozil is present at a concentration of approximately 5 mg/ml to 32 mg/ml and the liquid pharmaceutical composition is stable for at least 36 months at storage temperatures in the range of 2 to 40° C. The solvent may be an aqueous buffer having pH 10.5 (+/−0.5). The solvent may be a carbonate buffer having pH 10.5 (+/−0.5).

### BRIEF DESCRIPTION OF THE DRAWINGS

The following detailed description of the embodiments of the present invention will be better understood when read in conjunction with the appended drawings. It is understood, however, that the invention is not limited to the precise arrangements shown. In the drawings:

FIG. 1A shows results of a gemfibrozil assay for gemfibrozil in liquid pharmaceutical composition samples stored under various temperature and humidity conditions over 24 months while inverted.

FIG. 1B shows results of a gemfibrozil assay for gemfibrozil in the liquid pharmaceutical composition samples stored under various temperature and humidity conditions over 24 months while upright.

FIG. 2A shows results of an HPLC assay detecting gemfibrozil-related substances in liquid pharmaceutical composition samples stored under various temperature and humidity conditions over 24 months while inverted.

FIG. 2B shows results of an HPLC assay detecting gemfibrozil-related substances in the liquid pharmaceutical composition samples stored under various temperature and humidity conditions over 24 months while upright.

FIG. 3A shows pH values of the liquid pharmaceutical composition samples stored under various temperature and humidity conditions over 24 months while inverted.

FIG. 3B shows pH values of the liquid pharmaceutical composition samples stored under various temperature and humidity conditions over 24 months while upright.

FIG. 4A shows results of density measurements of liquid pharmaceutical composition samples stored under various temperature and humidity conditions over 24 months while inverted.

FIG. 4B shows density measurements of liquid pharmaceutical composition samples stored under various temperature and humidity conditions over 24 months while upright.

FIG. 5 shows a representative schematic of a method of making a liquid pharmaceutical composition comprising gemfibrozil.

FIG. 6A shows HPLC data from placebo control liquid pharmaceutical composition samples that have undergone freeze-thaw cycling.

FIG. 6B shows HPLC data of liquid pharmaceutical composition samples that have undergone freeze-thaw cycling.

FIG. 6C shows HPLC data from placebo control liquid pharmaceutical composition samples and liquid pharmaceutical composition samples that have not undergone freeze-thaw cycling.

FIG. 6D shows HPLC data from placebo control liquid pharmaceutical composition samples and liquid pharmaceutical composition samples that have undergone freeze-thaw cycling.

FIG. 7 shows a representative schematic of a method of making liquid pharmaceutical compositions comprising gemfibrozil and preservatives.

### DETAILED DESCRIPTION OF EMBODIMENTS

Certain terminology is used in the following description for convenience only and is not limiting. The phrase “at least one” followed by a list of two or more items such as “A, B, or C” or “A, B, and C” means any individual one of A, B, or C as well as any combination thereof. Terms such as “approximately,” “about,” “substantially” are construed as modifying terms defined by the circumstances and as understood by those skilled in the art. The degree of modification includes the degree of expected experimental error. The terms “liquid pharmaceutical composition” and “formulation” may be used interchangeably. The terms “storage” and “incubation” may be used interchangeably when referring to the duration of time that the liquid pharmaceutical composition is retained at a specified condition in order to evaluate the stability or other characteristics of the liquid pharmaceutical composition.

An embodiment includes a liquid pharmaceutical composition comprising, consisting essentially of, or consisting of gemfibrozil and an aqueous solvent. The gemfibrozil may have a concentration of approximately 5 mg/ml to 135 mg/ml in the liquid pharmaceutical composition. The gemfibrozil concentration may be 5 mg/ml to 10 mg/ml, 10 mg/ml to 15 mg/ml, 15 mg/ml to 20 mg/ml, 15 mg/ml to 30 mg/ml, 20 mg/ml to 25 mg/ml, 25 mg/ml to 30 mg/ml, 30 mg/ml to 35 mg/ml, 35 mg/ml to 40 mg/ml, 45 mg/ml to 50 mg/ml, 50 to 55 mg/ml, 55 mg/ml to 60 mg/ml, 60 mg/ml to 65 mg/ml, 65 mg/ml to 70 mg/ml, 70 mg/ml to 75 mg/ml, 75 mg/ml to 80 mg/ml, 80 mg/ml to 85 mg/ml, 85 mg/ml to 90 mg/ml, 90 mg/ml to 95 mg/ml, 95 mg/ml to 100 mg/ml, 100 mg/ml to 105 mg/ml, 105 mg/ml to 110 mg/ml, 110 mg/ml to 115 mg/ml, 115 mg/ml to 120 mg/ml, 120 mg/ml to 125 mg/ml, 125 mg/ml to 130 mg/ml, or 130 mg/ml to 135 mg/ml. The gemfibrozil concentration may be 5 mg/ml, 10 mg/ml, 15 mg/ml, 20 mg/ml, 25 mg/ml, 30 mg/ml, 35 mg/ml, 40 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, 100 mg/ml, 105 mg/ml, 110 mg/ml, 115 mg/ml, 120 mg/ml, 125 mg/ml, 130 mg/ml, or 135 mg/ml, or a concentration in a range between any two of the foregoing concentrations. The gemfibrozil may have a concentration selected from 1 mg/ml increments in the range of 5 mg/ml to 135 mg/ml (that is, the concentration may be 5 mg/ml, 6 mg/ml, 7 mg/ml, 8 mg/ml . . . 135 mg/ml). The gemfibrozil may have a concentration in a range selected from any two 1 mg/ml increments chosen from 5 mg/ml to 135 mg/ml (that is, the endpoints in the range may be chosen from 5 mg/ml, 6 mg/ml, 7 mg/ml, 8 mg/ml . . . 135 mg/ml). The gemfibrozil may have a concentration of approximately 5 mg/ml to 32 mg/ml in the liquid pharmaceutical composition. The gemfibrozil may have a concentration selected from 1 mg/ml increments in the range of 5 mg/ml to 32 mg/ml (that is, the solubility may be 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml, 26 mg/ml, 27 mg/ml, 28 mg/ml, 29 mg/ml, 30 mg/ml, 31 mg/ml, or 32 mg/ml). The gemfibrozil may be a concentration in a sub-range selected from any two 1 mg/ml increments in the range of 5 mg/ml to 32 mg/ml. The sub-range low endpoint may be selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 mg/ml. The sub-range high endpoint may be selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 mg/ml. The high endpoint selected is higher than the low endpoint selected. The gemfibrozil may have a concentration of approximately 5 mg/ml, 10 mg/ml, 12 mg/ml, 15 mg/ml, 20 mg/ml, 25 mg/ml, 30 mg/ml, or 32 mg/ml in the liquid pharmaceutical composition. The gemfibrozil may have a concentration of 5 mg/ml, 10 mg/ml, 12 mg/ml, 15 mg/ml, 20 mg/ml, 25 mg/ml, 30 mg/ml, or 32 mg/ml. The gemfibrozil may have a concentration of approximately 15 mg/ml.

The solvent may be an aqueous solvent having a pH of 10.5 (+/−0.5). The solvent may comprise, consist essentially of, or consist of a carbonate/bicarbonate buffer having a pH of 10.5 (+/−0.5). The carbonate may be sodium carbonate. The solvent may comprise, consist essentially of, or consist of a sodium carbonate/bicarbonate buffer having a pH of 10.5 (+/−0.2).

The liquid pharmaceutical composition may have an ionic strength in the range of 50 mM to 100 mM. The ionic strength may be 50, 55, 60, 65, 70, 75, 80, 85, 90, 95, or 100 mM, or in a range between any two of 50, 55, 60, 65, 70, 75, 80, 85, 90, 95, and 100 mM.

The liquid pharmaceutical composition may further comprise at least one of a pharmaceutically acceptable excipient or a pharmaceutically acceptable carrier.

The excipient may include one or more of a taste modifier or a sweetener. The taste modifier may comprise, consists essentially of, or consists of at least one of a flavoring, a sweeting agent, a liquid cherry flavor, a liquid orange flavor, a liquid strawberry flavor, a liquid vanilla flavor, a powdered cherry flavor, a powered orange flavor, a powered strawberry flavor, a powered vanilla flavor. The taste modifier comprises a powdered cherry flavor. In an embodiment, the cherry flavor may be in the range of 0.03% w/w to 0.3% w/w in the liquid pharmaceutical composition. The cherry flavor may be present at 0.1% w/w or 0.3% w/w in the liquid pharmaceutical composition. The sweetener may comprise, consists essentially of, or consist of sucralose or sodium saccharin. The sweetener may comprise sodium saccharin. Sodium saccharin may be present in the liquid pharmaceutical composition in the range of 0.05% w/w to 0.2% w/w. In an embodiment, the sodium saccharin may be present in the liquid pharmaceutical composition at 0.05% w/w. The liquid pharmaceutical composition may comprise a taste modifier and a sweetener. The liquid pharmaceutical composition may comprise a cherry flavor and sodium saccharin. The cherry flavor and sodium saccharin may be present in the liquid pharmaceutical composition at 0.1% w/w and 0.05% w/w, respectively.

The pharmaceutically acceptable carrier may include any one or more agents selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, polyethoxylated castor oil, phosphate buffered saline (PBS), or any other suitable pharmaceutically acceptable carrier.

The liquid pharmaceutical composition comprising gemfibrozil may further comprise a preservative. The preservative may minimize microbial growth. The preservative may also enhance the self-preserving efficacy of the liquid pharmaceutical composition. The preservative may comprise, consist essentially of, or consist of at least one of disodium ethylenediaminetetraacetic acid (EDTA) or domiphen bromide. In an embodiment, the preservative may be disodium EDTA. The EDTA may be present at 0.2% w/w in the final liquid pharmaceutical composition. In an embodiment, the preservative may be domiphen bromide. The domiphen bromide may be present at 0.2% w/w in the final liquid pharmaceutical composition.

In an embodiment, the density of the liquid pharmaceutical composition comprising gemfibrozil may be approximately 1.0 g/cm³. The density of the liquid pharmaceutical composition comprising gemfibrozil may be 1.0 g/cm³. The density of the liquid pharmaceutical composition may be 0.95 g/cm³, 1.00 g/cm³, 1.05 g/cm³, or in a sub-range thereof.

The stability of the gemfibrozil in the liquid pharmaceutical composition may be such that greater than 90% of the gemfibrozil is stable following incubation of the liquid pharmaceutical composition for 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, or 36 months. The stability of the gemfibrozil in the liquid pharmaceutical composition may be such that greater than 90% of the gemfibrozil is stable following incubation of the liquid pharmaceutical composition for any of the selected time periods described in this paragraph or any sub-range of time between any of the selected time periods described in this paragraph. The stability of the gemfibrozil may be such that greater than 90% of the gemfibrozil is stable following incubation of the liquid pharmaceutical composition for sub-ranges of time including 1 day to 1 week, 1 week to 2 weeks, 2 weeks to 1 month, 1 month to 2 months, 2 months to 3 months, 3 months to 6 months, 6 months to 9 months, 9 months to 12 months, 12 months to 18 months, 18 months to 24 months, 24 months to 36 months, or in a sub-range thereof. The sub-range low endpoint may be selected from 1 day, 1 week, 2 weeks, 1 month, 2 months, 3 months, 6 months, 9 months, 12 months, 18 months, or 24 months, while the sub-range high endpoint may be selected from 1 week, 2 weeks, 1 month, 2 months, 3 months, 6 months, 9 months, 12 months, 18 months, 24 months, or 36 months, where the high endpoint selected is higher than the low endpoint selected. The stability of the gemfibrozil in the liquid pharmaceutical composition may be such that greater than 95% of the gemfibrozil is stable following incubation of the liquid pharmaceutical composition for any of the selected time periods described in this paragraph or any sub-range of time between any of the selected time periods described in this paragraph. The gemfibrozil may be greater than 90% stable in the liquid pharmaceutical composition for up to 24 months including for any of the selected time periods described in this paragraph or any sub-range of time between any of the selected time periods described in this paragraph. The gemfibrozil may be greater than 90% stable in the liquid pharmaceutical composition for at least 24 months including for any of the selected time periods described in this paragraph or any sub-range of time between any of the selected time periods described in this paragraph. The gemfibrozil may be greater than 95% stable in the liquid pharmaceutical composition for up to 24 months including for any of the selected time periods described in this paragraph or any sub-range of time between any of the selected time periods described in this paragraph. The gemfibrozil may be greater than 95% stable in the liquid pharmaceutical composition for at least 24 months including for any of the selected time periods described in this paragraph or any sub-range of time between any of the selected time periods described in this paragraph. The gemfibrozil may be greater than 90% stable in the liquid pharmaceutical composition for up to 36 months including for any of the selected time periods described in this paragraph or any sub-range of time between any of the selected time periods described in this paragraph. The gemfibrozil may be greater than 90% stable in the liquid pharmaceutical composition for at least 36 months including for any of the selected time periods described in this paragraph or any sub-range of time between any of the selected time periods described in this paragraph. The gemfibrozil may be greater than 95% stable in the liquid pharmaceutical composition for up to 36 months including for any of the selected time periods described in this paragraph or any sub-range of time between any of the selected time periods described in this paragraph. The gemfibrozil may be greater than 95% stable in the liquid pharmaceutical composition for at least 36 months including for any of the selected time periods described in this paragraph or any sub-range of time between any of the selected time periods described in this paragraph. In an embodiment, the liquid pharmaceutical composition is stored or incubated is amber glass vials.

The stability of the gemfibrozil in the liquid pharmaceutical composition may be such that 90% to 100% is stable and free from degradation. The percent of the gemfibrozil in the liquid pharmaceutical composition may be stable and free from degradation in 0.5% increments in the range of 90% to 100% (that is, the percent may be 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100%. The percent may be in a sub-range with a low endpoint selected from 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5%, and a high endpoint selected from 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100%, where the low endpoint selected is lower than the high endpoint selected.

In an embodiment, the stability of the gemfibrozil in the liquid pharmaceutical composition may be such that greater than 90% of the gemfibrozil is stable following incubation of the liquid pharmaceutical composition at 2-8° C., 10° C., 15° C., ambient temperature, 20° C., 25° C., 30° C., 35° C., 40° C., 45° C., 50° C., 55° C., 60° C., 65° C., or in a sub-range thereof, The sub-range may be selected from one of 2 to 8° C., 8 to 10° C., 10 to 15° C., 15 to 20° C., 20 to 25° C., 25 to 30° C., 30 to 35° C., 35 to 40° C., 40 to 45° C., 45 to 50° C., 50 to 55° C., 55 to 60° C., or 60 to 65° C. The sub-range low endpoint may be selected from 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45 C, 46° C., 47° C., 48° C., 49° C., 50° C., 51° C., 52° C., 53° C., 54° C., 55 C, 56° C., 57° C., 58° C., 59° C., 60° C., 61° C., 62° C., 63° C., or 64° C., while the sub-range high endpoint may be selected from 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55 C, 56° C., 57° C., 58° C., 59° C., 60° C., 61° C., 62° C., 63° C., 64° C., or 65° C., where the high endpoint selected is higher than the low endpoint selected. In an embodiment, the stability of the gemfibrozil in the liquid pharmaceutical composition may be such that greater than 95% of the gemfibrozil is stable following incubation for any of the time periods described herein or any of the temperatures described in this paragraph. In an embodiment, the gemfibrozil in the liquid pharmaceutical composition may be such that greater than 90% of the gemfibrozil is stable following incubation at 2 to 8° C., 15° C., ambient temperature, 25° C., or 40° C. for any of the storage durations described herein. In an embodiment, the gemfibrozil in the liquid pharmaceutical composition may be such that greater than 95% of the gemfibrozil is stable following incubation at 2 to 8° C., 15° C., ambient temperature, 25° C., or 40° C. for any of the storage durations described herein.

The stability of the gemfibrozil in the liquid pharmaceutical composition may be such that greater than 90% of the gemfibrozil is stable following incubation of the liquid pharmaceutical composition for up to 18 months at temperatures greater than 2 to 8° C. but less than or equal to 25° C. The stability of the gemfibrozil in the liquid pharmaceutical composition may be such that greater than 90% of the gemfibrozil is stable following incubation of the liquid pharmaceutical composition for up to 24 months at temperatures greater than 2 to 8° C. but less than or equal to 25° C. The stability of the gemfibrozil in the liquid pharmaceutical composition may be such that greater than 90% of the gemfibrozil is stable following incubation of the liquid pharmaceutical composition for up to 36 months at temperatures greater than 2 to 8° C. but less than or equal to 25° C. The stability of the gemfibrozil in the liquid pharmaceutical composition may be such that greater than 90% of the gemfibrozil is stable following incubation of the liquid pharmaceutical composition for up until 18 months at 2 to 8° C. The stability of the gemfibrozil in the liquid pharmaceutical composition may be such that greater than 90% of the gemfibrozil is stable following incubation of the liquid pharmaceutical composition for up until 24 months at 2 to 8° C. The stability of the gemfibrozil in the liquid pharmaceutical composition may be such that greater than 90% of the gemfibrozil is stable following incubation of the liquid pharmaceutical composition for up until 36 months at 2 to 8° C. The stability of the gemfibrozil in the liquid pharmaceutical composition may be such that greater than 90% of the gemfibrozil is stable following incubation of the liquid pharmaceutical composition for up until 18 months at 25° C. The stability of the gemfibrozil in the liquid pharmaceutical composition may be such that greater than 90% of the gemfibrozil is stable following incubation of the liquid pharmaceutical composition for up until 24 months at 25° C. The stability of the gemfibrozil in the liquid pharmaceutical composition may be such that greater than 90% of the gemfibrozil is stable following incubation of the liquid pharmaceutical composition for up until 36 months at 25° C. The stability of the gemfibrozil in the liquid pharmaceutical composition may be such that greater than 90% of the gemfibrozil is stable following incubation of the liquid pharmaceutical composition for up until 6 months at 40° C. In an embodiment, the liquid pharmaceutical composition comprising gemfibrozil may be stored inverted. In an embodiment, the liquid pharmaceutical composition comprising gemfibrozil may be stored upright.

The stability of the gemfibrozil in the liquid pharmaceutical composition may be determined by conducting a gemfibrozil assay using HPLC. The gemfibrozil assay may be conducted on samples from any time point above throughout the duration of the storage conditions described herein. The time points may be 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range selected from any two of the foregoing. The storage conditions may include a storage temperature of 2-8° C., 25° C., 40° C., or in a sub-range thereof.

In an embodiment, the gemfibrozil assay results of the liquid pharmaceutical composition comprising gemfibrozil may not change throughout the time period and storage conditions referenced herein, including at least 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof, and including at least storage temperature of 2-8° C., 10° C., 15° C., ambient temperature, 20° C., 25° C., or in a sub-range thereof. In an embodiment, the gemfibrozil assay results of the liquid pharmaceutical composition comprising gemfibrozil may change but meet specification limits and stay within alarm limits throughout the time period and storage conditions referenced herein, including at least 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof, and including at least storage temperature of 2-8° C., 10° C., 15° C., ambient temperature, 20° C., 25° C., or in a sub-range thereof.

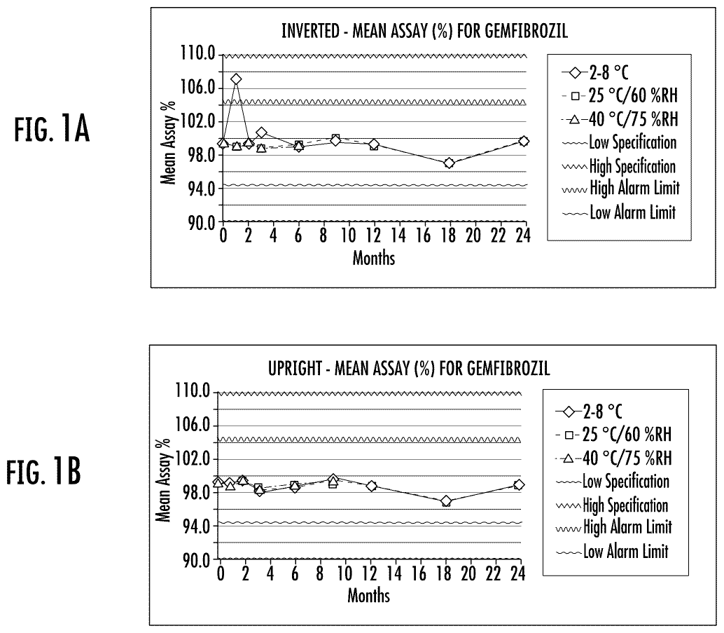

In an embodiment, the gemfibrozil assay results of the liquid pharmaceutical composition comprising gemfibrozil may be represented as shown in FIG. 1A and FIG. 1B.

The stability of the gemfibrozil in the liquid pharmaceutical composition may be determined by evaluating the liquid pharmaceutical composition for the formation of gemfibrozil-related substances. The formation of gemfibrozil-related substances may also be evaluated using HPLC to assess whether the gemfibrozil peak(s) are decreasing and/or whether new substances are present in the liquid pharmaceutical composition.

The formation of gemfibrozil-related substances may be evaluated on samples from any time point throughout the duration of the storage conditions described herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof. The storage conditions are those referred to herein and include at least storage temperature of 2-8° C., 25° C., 40° C., or in a sub-range thereof. Formation of gemfibrozil-related substances from the initial timepoint throughout the duration of the time periods described herein may not occur. Formation of gemfibrozil-related substances from the initial timepoint throughout the duration of the time periods described herein may not change. Formation of gemfibrozil-related substances from the initial timepoint throughout the duration of the time periods described herein may change slightly but may be within the alarm limits of the study (total impurities should have an increase of <1.00%, and any individual impurities should have an increase<0.20% of the correspondence peak area).

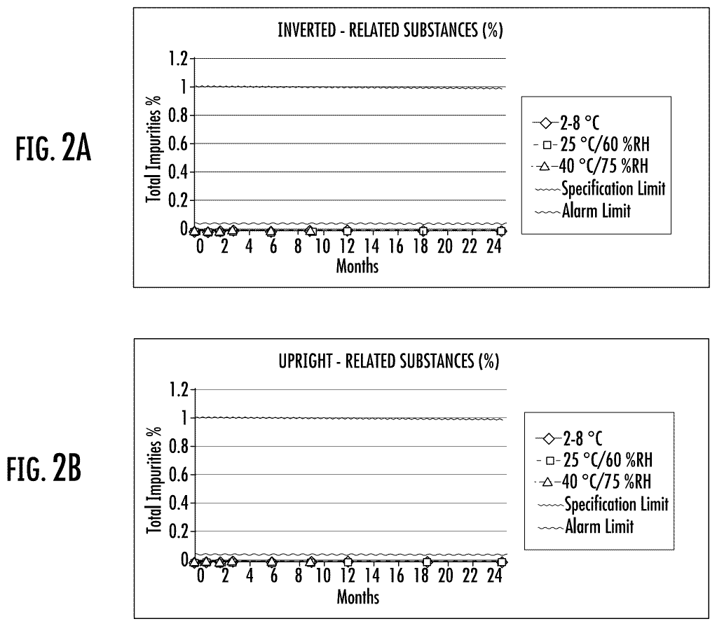

In an embodiment, the assay results of the liquid pharmaceutical composition for related substances of gemfibrozil may be represented as shown in FIG. 2A and FIG. 2B.

The liquid pharmaceutical composition comprising gemfibrozil may be stable.

The appearance of the liquid pharmaceutical composition comprising gemfibrozil may be determined by evaluating at least one of the color of the solution, whether crystalline precipitate forms, and whether the solution is clear or cloudy. These features may be compared with the initial timepoint (after preparation of the liquid pharmaceutical composition) to determine if any change occurs during storage. The presence of a crystalline precipitate, the color, and the transparency of the liquid pharmaceutical composition may be evaluated with user visualization. Acceptable changes within the alarm limits of the study include a colorless to pale yellow color, a clear solution, and a solution free from crystalline precipitate that does not re-dissolve upon warming at ambient temperature prior to use.

The liquid pharmaceutical composition comprising gemfibrozil may be free from crystalline precipitate over the time period of storage referenced herein including at least 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof. The liquid pharmaceutical composition comprising gemfibrozil may be free from crystalline precipitate formation under storage conditions at all temperatures referenced herein, including at least storage temperature of 2-8° C., 10° C., 15° C., ambient temperature, 20° C., 25° C., 30° C., 35° C., 40° C., or in a sub-range thereof. The liquid pharmaceutical composition comprising gemfibrozil may be free from crystalline precipitate formation for up to 24 months of storage at 2-8° C. or 25° C. The liquid pharmaceutical composition comprising gemfibrozil may be free from crystalline precipitate formation for up to 36 months of storage at 2-8° C. or 25° C. The liquid pharmaceutical composition comprising gemfibrozil may be free from crystalline precipitate formation for up to 6 months of storage at 40° C. In an embodiment, the liquid pharmaceutical composition comprising gemfibrozil may be stored inverted. In an embodiment, the liquid pharmaceutical composition comprising gemfibrozil may be stored upright. In an embodiment, liquid pharmaceutical composition placebo to match gemfibrozil may be free from crystalline precipitate formation under storage conditions at all temperatures referenced herein, including at least storage temperature of 2-8° C., 25° C., and 40° C. for up to 24 months. In an embodiment, liquid pharmaceutical composition placebo to match gemfibrozil samples may be free from crystalline precipitate formation under storage conditions at all temperatures referenced herein, including at least storage temperature of 2-8° C., 25° C., and 40° C. for up to 36 months. In an embodiment, formation of crystalline precipitate in the liquid pharmaceutical composition comprising gemfibrozil may occur. These samples may be reconstituted and further storage can be continued.

The liquid pharmaceutical composition comprising gemfibrozil may have little to no changes in color over the time period of storage referenced herein including at least 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof. The liquid pharmaceutical composition may have little to no changes in color at temperatures referenced herein, including at least storage temperature of 2-8° C., 10° C., 15° C., ambient temperature, 20° C., 25° C., or in a sub-range thereof. The liquid pharmaceutical composition comprising gemfibrozil may be colorless over the time period and under the storage conditions referenced herein. The liquid pharmaceutical composition comprising gemfibrozil may be colorless for a period of time and then have a pale-yellow color over a later period of time. The liquid pharmaceutical composition comprising gemfibrozil may be pale yellow over the time period and under the storage conditions referenced herein. In an embodiment, the liquid pharmaceutical composition comprising gemfibrozil may be stored upright.

The pH of the liquid pharmaceutical composition comprising gemfibrozil may be consistent over the time period and under the storage conditions referenced herein. The pH of the liquid pharmaceutical composition comprising gemfibrozil may change slightly but be within the alarm limits of the study (+/−0.5 units) over the time period and under the storage conditions referenced herein. The pH of the liquid pharmaceutical composition comprising gemfibrozil may have a pH of 10.5 over the time period and under the storage conditions referenced herein. The pH of the liquid pharmaceutical composition comprising gemfibrozil may change slightly but be within the alarm limits of the study (+/−0.5 units) over the time period and under the storage conditions referenced herein. The pH of the liquid pharmaceutical composition comprising gemfibrozil may be 10.5 (+/−0.5 units) over the time period and under the storage conditions referenced herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and including but not limited to storage at temperatures of 2-8° C., 10° C., 15° C., ambient temperature, 20° C., 25° C., 30° C., 35° C., 40° C., or in a sub-range thereof. The pH of the liquid pharmaceutical composition comprising gemfibrozil may change slightly from the initial time point but remain within the alarm limits (pH 10.5+/−0.5 units) over the time period and under the storage conditions referenced herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and including but not limited to storage at temperatures of 2-8° C., 10° C., 15° C., ambient temperature, 20° C., 25° C., 30° C., 35° C., 40° C., or in a sub-range thereof. In an embodiment, the liquid pharmaceutical composition comprising gemfibrozil may be stored in an inverted or upright position.

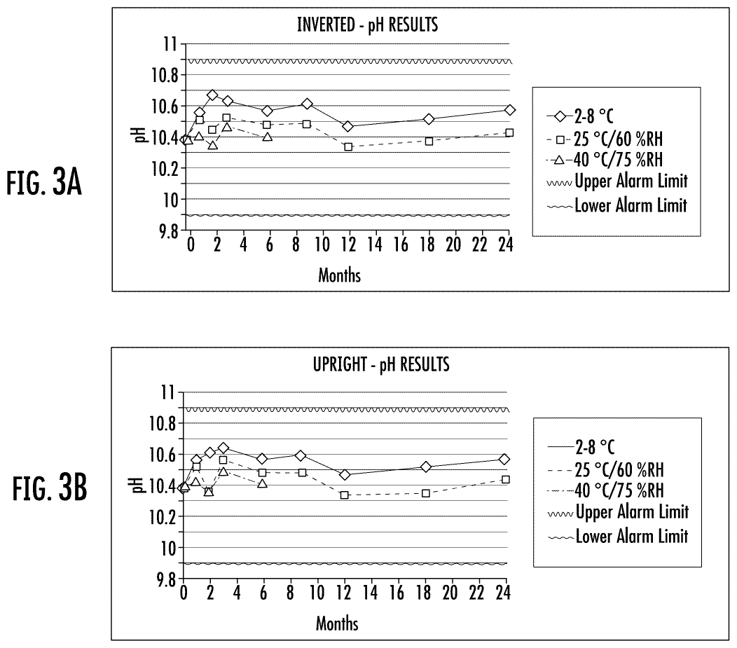

In an embodiment, the pH of the liquid pharmaceutical composition comprising gemfibrozil may be represented as shown in FIG. 3A and FIG. 3B over the time period indicated and storage conditions.

Density measurements of the liquid pharmaceutical composition comprising gemfibrozil may be consistent over the time period and under the storage conditions referenced herein. The density of the liquid pharmaceutical composition comprising gemfibrozil may be 1.01 g/cm³ over the time period and under the storage conditions referenced herein. The density of the liquid pharmaceutical composition comprising gemfibrozil may change slightly but be within (+/−0.05 g/cm³) over the time period and under the storage conditions referenced herein. The time period and the storage conditions referenced herein, may include but not be limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and include but not be limited to storage temperatures of 2-8° C., 10° C., 15° C., ambient temperature, 20° C., 25° C., 30° C., 35° C., 40° C., or in a sub-range thereof. In an embodiment, the liquid pharmaceutical composition comprising gemfibrozil may be stored in an inverted or upright position.

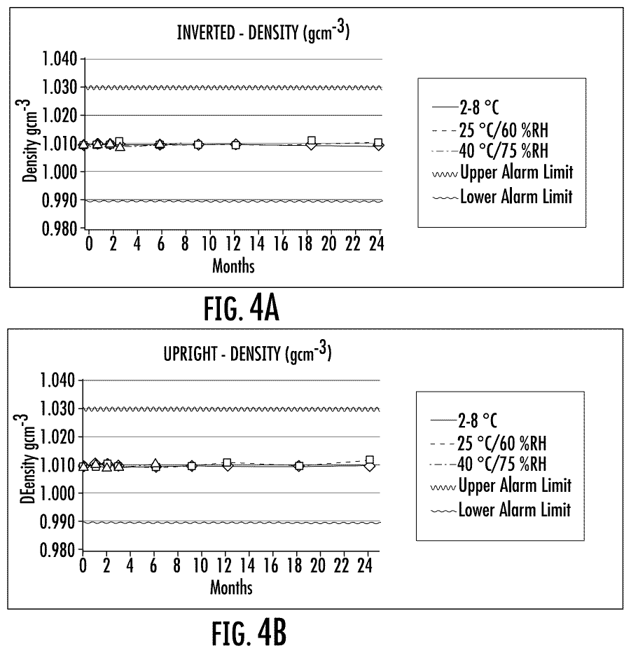

In an embodiment, the density of the liquid pharmaceutical composition comprising gemfibrozil may be represented as shown in FIG. 4A and FIG. 4B over the time period indicated and storage conditions.

The liquid pharmaceutical composition comprising gemfibrozil may also be evaluated to assess self-preservation ability of the composition by determining the microbial purity and preservative efficacy of the samples following storage under specified conditions.

Microbial purity may be determined by assessing the potential growth of Escherichia Coli (E. coli), total aerobic microbial count (TAMC), and total yeast/mold count (TYMC) according to current Ph Eur 9.8 (2.6.12, 2.6.13) and USP 42 <61><62> test method for total count and detection of specified organisms in active formulations of the liquid pharmaceutical composition. The acceptable standards that should be met by the pharmaceutical liquid composition comprising gemfibrozil should be the absence of E. Coli in 1 ml, less than 10 cfu/ml TAMC, and less than 10 cfu/ml TYMC according to current USP 42 <61><62> test method for total count and detection of specified organisms. Total aerobic microbial count, total E. coli count, and the total combined yeast and mold count of the liquid pharmaceutical composition comprising gemfibrozil may be within the alarm limits of the study over the time period and storage conditions referenced herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and including but not limited to storage at temperatures of 2-8° C., 10° C., 15° C., ambient temperature, 20° C., 25° C., 30° C., 35° C., 40° C., or in a sub-range thereof. In an embodiment, the liquid pharmaceutical composition comprising gemfibrozil may be stored in an inverted or upright position.

Preservative efficacy may be determined by assessing potential growth rates of bacteria and yeast/mold over time according to current USP 42<51> test method for total count and detection of specified organisms in active formulations of the liquid pharmaceutical composition over time. The acceptable standards that should be met by the pharmaceutical liquid composition comprising gemfibrozil should be a bacterial growth rate≥1 log reduction from 0 days to 14 days, with no increase from 14 days to 28 days, and no increase in yeast and molds from 0 days to 14 days and from 0 days to 28 days. Total bacterial count, and the total combined yeast and mold count of the liquid pharmaceutical composition comprising gemfibrozil between any two measured time points may be within the alarm limits of the study over the time period and storage conditions referenced herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and including but not limited to storage at temperatures of 2-8° C., 10° C., 15° C., ambient temperature, 20° C., 25° C., 30° C., 35° C., 40° C., or in a sub-range thereof. In an embodiment, the liquid pharmaceutical composition comprising gemfibrozil may be stored in an inverted or upright position.

Preservative efficacy may be determined by assessing for the presence of Burkholderia capacia complex (BCC) over time according to current USP <60> test method for total count and detection of specified organisms in active formulations of the liquid pharmaceutical composition over time. Total BCC count in the liquid pharmaceutical composition comprising gemfibrozil may be within the alarm limits of the study over the time period and storage conditions referenced herein, including but not limited to 1 day, 1 week, 2 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19, months, 20 months, 21 months, 22 months, 23 months, 24 months, 25 months, 26 months, 27 months, 28 months, 29 months, 30 months, 31 months, 32 months, 33 months, 34 months, 35 months, 36 months, or in a sub-range thereof and including but not limited to storage at temperatures of 2-8° C., 10° C., 15° C., ambient temperature, 20° C., 25° C., 30° C., 35° C., 40° C., or in a sub-range thereof. In an embodiment, the liquid pharmaceutical composition comprising gemfibrozil may be stored in an inverted or upright position.

### Uses

The liquid pharmaceutical composition may be for treating a lysosomal storage disorder, Neuronal Ceroid Lipofuscinoses, or a neurodegenerative disorder. The lysosomal storage disorder may be any one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis. The neurodegenerative disorder may be Alzheimer's disease, Parkinson's disease, Dementia with Lewy Bodies, Multiple System Atrophies, Multiple Sclerosis, Huntington's disease, or Amyotrophic Lateral Sclerosis.

The liquid pharmaceutical composition may be used in the treatment of a lysosomal storage disorder, Neuronal Ceroid Lipofuscinoses, or a neurodegenerative disorder. The lysosomal storage disorder may be any one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis. The neurodegenerative disorder may be Alzheimer's disease, Parkinson's disease, Dementia with Lewy Bodies, Multiple System Atrophies, Multiple Sclerosis, Huntington's disease, or Amyotrophic Lateral Sclerosis.

The liquid pharmaceutical composition may be used in the preparation of a medicament for treating a lysosomal storage disorder, Neuronal Ceroid Lipofuscinoses, or a neurodegenerative disorder. The lysosomal storage disorder may be any one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis. The neurodegenerative disorder may be Alzheimer's disease, Parkinson's disease, Dementia with Lewy Bodies, Multiple System Atrophies, Multiple Sclerosis, Huntington's disease, or Amyotrophic Lateral Sclerosis.

### Methods of Making

An embodiment comprises a method of making a liquid pharmaceutical composition comprising gemfibrozil. The method may include preparing gemfibrozil slurry. Preparing a gemfibrozil slurry may comprise adding gemfibrozil to water and mixing to form the gemfibrozil slurry. The mixing may be performed with a spatula.

The method of making may comprise mixing gemfibrozil or a gemfibrozil slurry in an aqueous buffer. The buffer may have a pH of 10 to 11. The buffer may be a carbonate/bicarbonate buffer having a pH of 10.5 (+/−0.5). The buffer may be a sodium carbonate/bicarbonate buffer having a pH of 10.5 (+/−0.5).

The buffer may be prepared by mixing the sodium carbonate with water. The mixing may occur by stirring, spinning, vortexing, or other means known in the art for at least 1 minute or until the sodium carbonate is dissolved. In an embodiment, the sodium carbonate and water are mixed by spinning at a low speed to avoid splashing or aeration. The water-sodium carbonate solution may be mixed and/or maintained at a temperature of 15° C., ambient temperature, 20° C., 25° C., 30° C., 35° C., 40° C., or in a sub-range thereof. Sodium hydrogen carbonate may be further added to the water-sodium carbonate solution and mixed for at least 1 minute, 2 minutes, or any interval in between either of these ranges, or until the sodium hydrogen carbonate is dissolved. This solution may be mixed and/or maintained as described in this paragraph. In an embodiment, the sodium hydrogen carbonate may be added first and the sodium carbonate may be added second. Sodium hydroxide may be added to the sodium carbonate/bicarbonate solution and mixed for approximately 30 seconds, two minutes, or any interval in between either of these ranges or for any time known in the art sufficient for such mixing. In an embodiment, the sodium carbonate/bicarbonate solution is mixed by spinning at a low speed to avoid splashing or aeration. The sodium hydroxide added may be a 1 M solution. The sodium carbonate/bicarbonate solution comprising sodium hydroxide may be heated to 40° C. (+/−0.3). Heating may occur with the use of a silicone heater belt around the bulk vessel housing the solution, or using other means known in the art for warming solutions. The temperature may be monitored using a temperature probe. In an embodiment, the bulk vessel is a stainless-steel vessel.

The method may comprise adding sodium saccharin powder to a bulk vessel comprising the buffer. Mixing of the sodium saccharin may be done until fully dissolved, for 1 minute, for two minutes, or any interval in between either of these ranges, or for any other time known in the art for mixing such solutions.

The method may comprise adding cherry flavor powder to the bulk vessel containing the solution. Mixing of the cherry flavor powder may be done until fully dissolved, for 1 minute, 2 minutes, 5 minutes, 10 minutes, or any interval in between either of these ranges or for any other time known in the art for mixing such solutions. In an embodiment, the cherry flavor powder may be added first and the sodium saccharin may be added second. In an embodiment, the solution is maintained at 40° C. (+/−0.3).

In an embodiment, the gemfibrozil slurry may be added to the buffer, which may be a sodium carbonate/bicarbonate solution. The solution may further comprise an excipient, e.g., cherry flavor and/or sodium saccharin. The solution may be mixed for 10 minutes, 14 minutes, 20 minutes, 25 minutes, or any interval in between either of these ranges, or at least until all material is fully dissolved. In an embodiment, the mixing speed is 695 rpm, or any suitable missing speed commonly known in the art for such mixing. The mixing may also include heating the mixture to 40° C. (+/−0.3). Following mixing of the gemfibrozil, the solution may be removed from the heat source and allowed to cool. Additional water may be added and further mixing performed until the solution cools to <25° C. The pH of the solution may be taken and adjusted with either sodium hydroxide or hydrochloric acid while mixing to maintain a pH 10.5 (+/−0.05). The solution may then be brought to volume with water and stirred. The solution may be cooled overnight to <25° C.

An embodiment of the method of making comprises mixing the carbonate buffer having pH 10.5 (sodium carbonate+sodium bicarbonate+water+1.0 M NaOH), sodium saccharin, and black cherry flavor at room temperature in a stainless-steel vessel until the solids dissolved. The solution was then heated to 65° C. and gemfibrozil was added and mixed at 65° C. until the solids dissolved. Samples were cooled to 20° C. to 25° C. while stirring, the pH was adjusted to 10.5+/−0.05 with NaOH and the sample was brought to volume (1000 ml in this case) with either water or carbonate buffer having pH 10.5.

The time to dissolve gemfibrozil in the liquid pharmaceutical solution and reach a concentration matching an above-referenced gemfibrozil concentration may range from immediate upon mixing to 7 days in solution. The time may be immediate to 1 minute, 1 minute to 5 minutes, 5 minutes to 10 minutes, 10 minutes to 15 minutes, 15 minutes to 20 minutes, 20 minutes to 30 minutes, 30 minutes to one hour, one hour to 2 hours, 2 hours to 5 hours, 5 hours to 10 hours, 10 hours to 24 hours, 24 hours to 2 days, 2 days to 5 days, or 5 days to 7 days. The time may be approximately 1 minute, 5 minutes, 10 minutes, 15 minutes, 20 minutes, 30 minutes, one hour, 2 hours, 5 hours, 10 hours, 24 hours, 2 days, 5 days, or 7 days, or in a range between any two of the foregoing. In an embodiment, the time to dissolve gemfibrozil in the liquid pharmaceutical solution and reach a concentration matching an above-referenced gemfibrozil concentration values may be 20 minutes.

In an embodiment, the time to reach and/or the ability to reach the above-referenced gemfibrozil concentration values in the liquid pharmaceutical composition may be affected by the temperature of the liquid formulation. In an embodiment, the solubility of gemfibrozil and its ability to remain at the above-referenced concentration values in the liquid pharmaceutical composition may be affected by the temperature. In an embodiment, the formulations may be mixed at 2-8° C., 10° C., 15° C., ambient temperature, 20° C., 25° C., 30° C., 35° C., 40° C., 45° C., 50° C., 55° C., 60° C., 65° C. In an embodiment, the formulations may be mixed at a temperature in a range with a low endpoint and a high endpoint. The low endpoint may be selected from 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45 C, 46° C., 47° C., 48° C., 49° C., 50° C., 51° C., 52° C., 53° C., 54° C., 55 C, 56° C., 57° C., 58° C., 59° C., 60° C., 61° C., 62° C., 63° C., or 64° C., while the high endpoint may be selected from 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45 C, 46° C., 47° C., 48° C., 49° C., 50° C., 51° C., 52° C., 53° C., 54° C., 55 C, 56° C., 57° C., 58° C., 59° C., 60° C., 61° C., 62° C., 63° C., 64° C., or 65° C. The high endpoint selected is higher than the low endpoint selected. The formulations may be mixed at 40° C. The formulations may be mixed at ambient temperature. The stability of the gemfibrozil in the formulation may be such that greater than 90% or greater than 95% of the gemfibrozil is stable following incubation of the gemfibrozil formulation for any of the time periods described herein or any of the temperatures described in this paragraph.

The time to reach and/or the ability to reach the gemfibrozil solubility and above-referenced concentration values may be affected by the mixing speed of the formulation. In an embodiment, the formulations may be mixed/stirred at 0 rpm, 50 rpm, 100 rpm, 150 rpm, 200 rpm, 210 rpm, 220 rpm, 230 rpm, 240 rpm, 250 rpm, 260 rpm, 270 rpm, 280 rpm, 290 rpm, 300 rpm, 350 rpm, 400 rpm, 450 rpm, 500 rpm, 550 rpm, 600 rpm, 650 rpm, 700 rpm, 750 rpm, or in a sub-range thereof.

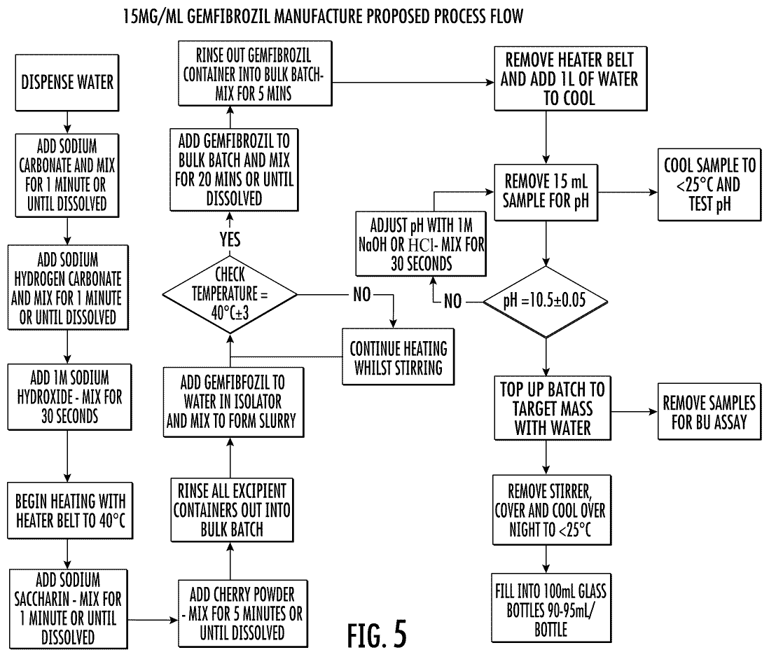

In an embodiment, the method of making a liquid pharmaceutical composition comprising gemfibrozil may be done according to the steps outlined in FIG. 5.

### Methods of Treating

An embodiment comprises a method of treating a subject. The method comprises administering a liquid pharmaceutical composition comprising gemfibrozil to a subject in need thereof. The liquid pharmaceutical composition comprising gemfibrozil may be one described herein.

The administering may be oral. The subject may be a patient. The subject may have a lysosomal storage disorder, a Neuronal Ceroid Lipofuscinoses, or a neurodegenerative disorder. The treating may be for ameliorating, curing or preventing a lysosomal storage disorder, a Neuronal Ceroid Lipofuscinoses, or a neurodegenerative disorder. The lysosomal storage disorder may be any one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, or Galactosialidosis. The neurodegenerative disorder may be Alzheimer's disease, Parkinson's disease, Dementia with Lewy Bodies, Multiple System Atrophies, Multiple Sclerosis, Huntington's disease, or Amyotrophic Lateral Sclerosis.

The subject may be mammal, a mouse, a dog, a horse, livestock, a human, a juvenile of any of the foregoing, a juvenile human. The subject in need thereof may include a patient who needs to lower systemic triglyceride levels. A subject in need thereof may also include a patient having lysosomal storage disorders including Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis, and Neuronal Ceroid Lipofuscinoses as well as neurodegenerative disorders including Alzheimer's disease, Parkinson's disease, Dementia with Lewy Bodies, Multiple System Atrophies, Multiple Sclerosis, Huntington's disease, and Amyotrophic Lateral Sclerosis. A subject in need thereof may also include a patient being treated with a form of a gemfibrozil tablet that has difficulty swallowing the tablet form of gemfibrozil.

Gemfibrozil, or liquid pharmaceutical composition comprising gemfibrozil, may be used either alone or in combination with other lysosomal storage disorder treatment drugs to enhance lysosomal-mediated waste clearance in certain cells in the brain. A method herein comprises administering a liquid pharmaceutical composition comprising gemfibrozil herein and one or more additional lysosomal storage disorder treatment drugs. The one or more additional lysosomal storage disorder treatment drugs may be selected from a lipid lowering drug, including but not limited to fenofibrate, clofibrate, bezafibrate, ciprofibrate and clinofibrate, Clofibrate, Bezafibrate, Ciprofibrate, or Clinobibrate; an anti-epilepsy drug, including but not limited to acetazolamide, benzodiazepine, cannabadiols, carbamazepine, clobazam, clonazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, sodium valproate, stiripentol, tiagabine, vigabatrin, or zonisamide; and other drugs known to treat lysosomal storage disorders.

While the present invention has been described in terms of its specific embodiments and specific examples, certain modifications and equivalents will be apparent to those skilled in the art and are intended to be included within the scope of the present invention.

### Embodiments of Detailed Description

1. A liquid pharmaceutical composition for the treatment of lysosomal storage disorders comprising, consisting essentially of, or consisting of gemfibrozil.

2. The liquid pharmaceutical composition of embodiment 1, further comprising a solvent.

3. The liquid pharmaceutical composition of any one or more of embodiments 1 or 2, where the solvent comprises, consists essentially of, or consists a buffer having a pH of 10 to 11. The pH may be 10, 10.1, 10.2, 10.3, 10.4, 10.5, 10.6, 10.7, 10.8, 10.9, or 11, or in a range between any two of 10, 10.1, 10.2, 10.3, 10.4, 10.5, 10.6, 10.7, 10.8, 10.9, and 11.

4. The liquid pharmaceutical composition of any one or more of embodiments 1 to 3, where the solvent comprises, consists essentially of, or consists of a sodium carbonate/bicarbonate buffer having a pH of 10.5 (+/−0.5).

5. The liquid pharmaceutical composition of any one or more of embodiments 1 to 4, wherein the pH of the liquid pharmaceutical composition is in the range of pH 10.3 to pH 10.7

6. The liquid pharmaceutical composition of any one or more of embodiments 1 to 5, wherein the pH of the liquid pharmaceutical composition is 10.5.

7. The liquid pharmaceutical composition of any one or more of embodiments 1 to 6, where the gemfibrozil is soluble in the pharmaceutical liquid composition at a final concentration of 5 mg/ml to 32 mg/ml.

8. The liquid pharmaceutical composition of any one or more of embodiments 1 to 7, where the gemfibrozil is stable for 24 months of storage.

9. The liquid pharmaceutical composition of any one or more of embodiments 1 to 8, where the gemfibrozil is stable for 36 months of storage.

10. The liquid pharmaceutical composition of any one or more of embodiments 1 to 9, where storage occurs at 2-8° C., 25° C., or 40° C.

11. The liquid pharmaceutical composition of any one or more of embodiments 1 to 10, where there is no detectable increase in gemfibrozil-related substances following 24 months in storage.

12. The liquid pharmaceutical composition of any one or more of embodiments 1 to 11, where the appearance of the composition does not change significantly following 24 months in storage.

13. The liquid pharmaceutical composition of any one or more of embodiments 1 to 12, where the liquid pharmaceutical composition is free from crystalline precipitate following 24 months in storage.

14. The liquid pharmaceutical composition of any one or more of embodiments 1 to 13, where the liquid pharmaceutical composition is clear following 24 months in storage.

15. The liquid pharmaceutical composition of any one or more of embodiments 1 to 14, where the liquid pharmaceutical composition is colorless following 24 months in storage.

16. The liquid pharmaceutical composition of any one or more of embodiments 1 to 15, where the liquid pharmaceutical composition is pale yellow following 24 months in storage.

17. The liquid pharmaceutical composition of any one or more of embodiments 1 to 17, where the liquid pharmaceutical composition is free from crystalline precipitate following 24 months in storage.

18. The liquid pharmaceutical composition of any one or more of embodiments 1 to 17, where the density of the composition does not change significantly following 24 months in storage.

19. The liquid pharmaceutical composition of any one or more of embodiments 1 to 18, where the liquid pharmaceutical composition has a pH of 10.5 (+/−0.5) following 24 months in storage.

20. The liquid pharmaceutical composition of any one or more of embodiments 1 to 19, where the liquid pharmaceutical composition has a pH of 10.5 (+/−0.5) following 24 months in storage at 2-8° C. or 25° C.

21. The liquid pharmaceutical composition of any one or more of embodiments 1 to 20, where microbial growth in the liquid pharmaceutical composition is prevented following 24 months in storage.

22. The liquid pharmaceutical composition of any one or more of embodiments 1 to 21, where preservative efficacy of the liquid pharmaceutical composition is maintained following 24 months in storage.

23. The liquid pharmaceutical composition of any one or more of embodiments 1 to 22, where the liquid pharmaceutical composition comprises a sweetener.

24. The liquid pharmaceutical composition of any one or more of embodiments 1 to 23, where the sweetener comprises, consists essentially of, or consists of sucralose or sodium saccharin.

25. The liquid pharmaceutical composition of any one or more of embodiments 1 to 24, where the sweetener comprises sodium saccharin.

26. The liquid pharmaceutical composition of any one or more of embodiments 1 to 17, where the sodium saccharin is present at a final concentration in the range of 0.02% to 0.2% weight/volume. The sodium saccharin concentration may be 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.2% (w/v), or in a range between any two of 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, and 0.2% (w/v).

27. The liquid pharmaceutical composition of any one or more of embodiments 1 to 26, where the liquid pharmaceutical composition comprises a taste modifier.

28. The liquid pharmaceutical composition of any one or more of embodiments 1 to 27, where the taste modifier comprises, consists essentially of, or consists of at least one of a flavoring, a liquid cherry flavor, or a powdered cherry flavor.

29. The liquid pharmaceutical composition of any one or more of embodiments 1 to 28, where the taste modifier is present at a final concentration in the range of 0.03% to 0.3% weight/volume. The taste modifier concentration may be 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, or 0.30% (w/v), or in a range between any two of 0.03, 0.04, 0.05, 0.06, 0.07, 0.08, 0.09, 0.10, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, and 0.30% (w/v).

30. The liquid pharmaceutical composition of any one or more of embodiments 1 to 29, further comprising a preservative.

31. The liquid pharmaceutical composition of embodiment 30, where the preservative is at least one of ethylenediaminetetraacetic acid (EDTA) or domiphen bromide.

32. The liquid pharmaceutical composition of any one or more of embodiments 1 to 31, where the ionic strength of the liquid pharmaceutical composition is in the range of 50 mM to 100 mM. The ionic strength may be 50, 55, 60, 65, 70, 75, 80, 85, 90, 95, or 100 mM, or in a range between any two of 50, 55, 60, 65, 70, 75, 80, 85, 90, 95, and 100 mM.

33. The liquid pharmaceutical composition of any one or more of embodiments 1 to 32, where the concentration of gemfibrozil is 15 mg/ml.

34. The liquid pharmaceutical composition of any one or more of embodiments 1 to 32, where the concentration of gemfibrozil is 15 mg/ml.

35. The liquid pharmaceutical composition of any one or more of embodiments 1 to 34, where the lysosomal storage disorders is at least one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis, and Neuronal Ceroid Lipofuscinoses as well as neurodegenerative disorders including Alzheimer's disease, Parkinson's disease, Dementia with Lewy Bodies, Multiple System Atrophies, Multiple Sclerosis, Huntington's disease, or Amyotrophic Lateral Sclerosis.

36. The liquid pharmaceutical composition of any one or more of embodiments 1 to 35, further comprising a pharmaceutically acceptable excipient.

37. A method of preparing a liquid pharmaceutical composition for treating a lysosomal storage disorder, comprising, consisting essentially of, or consisting of gemfibrozil.

38. The method of preparing a liquid pharmaceutical composition for treating the lysosomal storage disorder of embodiment 37, further comprising adding gemfibrozil to a solvent.

39. The method of preparing a liquid pharmaceutical composition for treating the lysosomal storage disorder of any one or more of embodiments 37 to 38, where the gemfibrozil is present in the pharmaceutical liquid composition at a final concentration of 5 mg/ml to 32 mg/ml.

40. The method of preparing a liquid pharmaceutical composition for treating the lysosomal storage disorder of any one or more of embodiments 37 to 39, where the gemfibrozil is present in the pharmaceutical liquid composition at a final concentration of 15 mg/ml.

41. The method of preparing a liquid pharmaceutical composition for treating the lysosomal storage disorder of any one or more of embodiments 37 to 40, where the gemfibrozil is present in the pharmaceutical liquid composition at a final concentration of 30 mg/ml.

42. The method of preparing a liquid pharmaceutical composition for treating the lysosomal storage disorder of any one or more of embodiments 37 to 41, where the pH of the liquid pharmaceutical composition is 10.5 (+/−0.5).

43. The method of preparing a liquid pharmaceutical composition for treating the lysosomal storage disorder of any one or more of embodiments 37 to 42, where the pH of the liquid pharmaceutical composition is 10.5 (+/−0.2).

44. The method of preparing a liquid pharmaceutical composition for treating the lysosomal storage disorder of any one or more of embodiments 37 to 43, where the solvent is a sodium carbonate/bicarbonate buffer having a pH of 10.5 (+/−0.5).

45. The method of preparing a liquid pharmaceutical composition for treating the lysosomal storage disorder of any one or more of embodiments 37 to 44, where the solvent is prepared by mixing the sodium carbonate and sodium bicarbonate with water, and adding sodium hydroxide.

46. The method of preparing a liquid pharmaceutical composition for treating the lysosomal storage disorder of any one or more of embodiments 37 to 45, where the solvent is heated to 40° C.

47. The method of preparing a liquid pharmaceutical composition for treating the lysosomal storage disorder of any one or more of embodiments 37 to 46, further comprising adding a sweetener to the liquid pharmaceutical composition.

48. The method of preparing a liquid pharmaceutical composition for treating the lysosomal storage disorder of any one or more of embodiments 37 to 47, further comprising mixing the sweetener with the liquid pharmaceutical composition by stirring at 40° C.

49. The method of preparing a liquid pharmaceutical composition for treating the lysosomal storage disorder of embodiment 48, where the sweetener is sodium saccharin.

50. The method of preparing a liquid pharmaceutical composition for treating the lysosomal storage disorder of any one or more of embodiments 48 to 49, further comprising passing the sodium saccharin through a 400 μM mesh prior to adding to the composition.

51. The method of preparing a liquid pharmaceutical composition for treating the lysosomal storage disorder of any one or more of embodiments 37 to 50, further comprising adding a taste modifier to the liquid pharmaceutical composition.

52. The method of preparing a liquid pharmaceutical composition for treating the lysosomal storage disorder of any one or more of embodiments 37 to 51, further comprising mixing the taste modifier with the liquid pharmaceutical composition by stirring at 40° C.

53. The method of preparing a liquid pharmaceutical composition for treating the lysosomal storage disorder of embodiments 52, where the taste modifier is at least one of a flavoring, a liquid cherry flavor, or a powdered cherry flavor.

54. The method of preparing a liquid pharmaceutical composition for treating the lysosomal storage disorder of any one or more of embodiments 37 to 53, where the gemfibrozil is mixed with water to form a slurry prior to mixing with the sodium carbonate/bicarbonate solvent.

55. The method of preparing a liquid pharmaceutical composition for treating the lysosomal storage disorder of any one or more of embodiments 37 to 54, further comprising mixing the gemfibrozil slurry with the solvent at 40° C. at least until the gemfibrozil dissolves.

56. The method of preparing a liquid pharmaceutical composition for treating the lysosomal storage disorder of any one or more of embodiments 37 to 55, further comprising mixing the gemfibrozil slurry with the solvent at 65° C. at least until the gemfibrozil dissolves.

57. The method of preparing a liquid pharmaceutical composition for treating the lysosomal storage disorder of any one or more of embodiments 37 to 56, where the mixing of the gemfibrozil slurry with the solvent comprises stirring in the range of 230 rpm to 695 rpm.

58. The method of preparing a liquid pharmaceutical composition for treating the lysosomal storage disorder of any one or more of embodiments 37 to 58, where the mixing comprises stirring for a time ranging from 1 minute to 20 minutes, or at least until the gemfibrozil is dissolved.

59. The method of preparing a liquid pharmaceutical composition for treating the lysosomal storage disorder of any one or more of embodiments 37 to 58, where the mixing of the gemfibrozil slurry with the solvent occurs at a range of temperatures from ambient temperature to approximately 65° C.

60. The method of preparing a liquid pharmaceutical composition for treating the lysosomal storage disorder of any one or more of embodiments 37 to 58, where the mixing of the gemfibrozil slurry with the solvent occurs at a temperature of approximately 40° C.

61. The method of preparing a liquid pharmaceutical composition for treating the lysosomal storage disorder of any one or more of embodiments 37 to 60, further comprising adding a preservative.

62. The method of preparing a liquid pharmaceutical composition for treating the lysosomal storage disorder of embodiment 61, where the preservative is at least one of ethylenediaminetetraacetic acid (EDTA) or domiphen bromide.

63. The method of preparing a liquid pharmaceutical composition for treating the lysosomal storage disorder of any one or more of embodiments 37 to 62, where the solvent is a sodium carbonate/bicarbonate buffer having a pH of 10.5 (+/−0.5) and the pH of the liquid pharmaceutical composition maintains a pH of 10.5 (+/−0.5).

64. The method of preparing a liquid pharmaceutical composition for treating the lysosomal storage disorder of any one or more of embodiments 37 to 63, where the gemfibrozil is stable for 24 months of storage.

65. The method of preparing a liquid pharmaceutical composition for treating the lysosomal storage disorder of any one or more of embodiments 37 to 64, where the gemfibrozil is stable for 36 months of storage.

66. The method of preparing a liquid pharmaceutical composition for treating the lysosomal storage disorder of any one or more of embodiments 37 to 65, where the liquid pharmaceutical composition is stable for 24 months of storage.

67. The method of preparing a liquid pharmaceutical composition for treating the lysosomal storage disorder of any one or more of embodiments 37 to 66, where the liquid pharmaceutical composition is stable for 36 months of storage.

68. The method of preparing a liquid pharmaceutical composition for treating the lysosomal storage disorder of any one or more of embodiments 1 to 68, where the liquid pharmaceutical composition is free from crystalline precipitate following 24 months in storage.

69. The method of preparing a liquid pharmaceutical composition for treating the lysosomal storage disorder of any one or more of embodiments 1 to 68, where the liquid pharmaceutical composition is free from crystalline precipitate following 36 months in storage.

70. The method of preparing a liquid pharmaceutical composition for treating the lysosomal storage disorder of any one or more of embodiments 1 to 69, where the liquid pharmaceutical composition is clear following 24 months in storage.

71. The method of preparing a liquid pharmaceutical composition for treating the lysosomal storage disorder of any one or more of embodiments 1 to 70, where the liquid pharmaceutical composition is clear following 36 months in storage.

72. The method of preparing a liquid pharmaceutical composition for treating the lysosomal storage disorder of any one or more of embodiments 1 to 71, where the liquid pharmaceutical composition is colorless following 24 months in storage.

73. The method of preparing a liquid pharmaceutical composition for treating the lysosomal storage disorder of any one or more of embodiments 1 to 72, where the liquid pharmaceutical composition is colorless following 36 months in storage.

74. The method of preparing a liquid pharmaceutical composition for treating the lysosomal storage disorder of any one or more of embodiments 1 to 73, where the liquid pharmaceutical composition is pale yellow following 24 months in storage.

75. The method of preparing a liquid pharmaceutical composition for treating the lysosomal storage disorder of any one or more of embodiments 1 to 74, where the liquid pharmaceutical composition is pale yellow following 36 months in storage.

76. The method of preparing a liquid pharmaceutical composition for treating the lysosomal storage disorder of any one or more of embodiments 37 to 75, where microbial growth in the liquid pharmaceutical composition is prevented following 24 months in storage.

77. The method of preparing a liquid pharmaceutical composition for treating the lysosomal storage disorder of any one or more of embodiments 37 to 76, where microbial growth in the liquid pharmaceutical composition is prevented following 36 months in storage.

78. The method of preparing a liquid pharmaceutical composition for treating the lysosomal storage disorder of any one or more of embodiments 37 to 77, where preservative efficacy of the liquid pharmaceutical composition is maintained following 24 months in storage.

79. The method of preparing a liquid pharmaceutical composition for treating the lysosomal storage disorder of any one or more of embodiments 37 to 78, where preservative efficacy of the liquid pharmaceutical composition is maintained following 36 months in storage.

80. The method of preparing a liquid pharmaceutical composition for treating the lysosomal storage disorder of any one or more of embodiments 37 to 79, where storage occurs at temperatures of 2-8° C., 25° C., or 40° C., or any temperature in between 2-25° C. or 2-40° C.

81. The method of preparing a liquid pharmaceutical composition for treating the lysosomal storage disorder of any one or more of embodiments 37 to 80, where storage occurs at temperatures of 2-8° C. or 25° C.

82. The method of preparing a liquid pharmaceutical composition for treating the lysosomal storage disorder of any one or more of embodiments 37 to 81, where the lysosomal storage disorders is any one of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, and Galactosialidosis, and Neuronal Ceroid Lipofuscinoses as well as neurodegenerative disorders including Alzheimer's disease, Parkinson's disease, Dementia with Lewy Bodies, Multiple System Atrophies, Multiple Sclerosis, Huntington's disease, or Amyotrophic Lateral Sclerosis.

83. A method of treating a subject having a lysosomal storage disorder comprising, consisting essentially of, or consisting of administering the liquid pharmaceutical composition of any one or more of embodiments 1 to 36 or the liquid pharmaceutical composition made by any one or more of embodiments 37 to 82 to the subject.

84. The method of treating a subject having a lysosomal storage disorder of embodiment 65 where treating comprises, consists essentially of, or consists of curing, slowing down, or halting the progression of the lysosomal storage disorder.

85. The method of treating a subject having the lysosomal storage disorder of any one or more of embodiment 83 to 84, where the administering comprises a route of administration comprising one or more routes selected from the group consisting of oral, sublingual, sublabial, buccal, and transmucosal.

86. The method of treating a subject having the lysosomal storage disorder of any one or more of embodiment 83 to 85, where the concentration of the gemfibrozil in the liquid pharmaceutical composition is in the range of 5 mg/ml to 32 mg/ml.

87. The method of treating a subject having the lysosomal storage disorder of embodiment 86, where the concentration of the gemfibrozil in the liquid pharmaceutical composition is 15 mg/ml.

88. The method of treating a subject having the lysosomal storage disorder of embodiment 86, where the concentration of the gemfibrozil in the liquid pharmaceutical composition is 30 mg/ml.

89. The method of treating a subject having the lysosomal storage disorder of any one or more of embodiment 83 to 88, where the dose of gemfibrozil used to treat the subject is in the range of 600 mg to 1800 mg per day. The dose may be 600, 700, 800, 900, 1000, 1100, 1200, 1300, 1400, 1500, 1600, 1700, or 1800, mg per day, or in a range between any two of the foregoing.

90. The method of treating a subject having the lysosomal storage disorder of any one or more of embodiment 83 to 89, further comprising co-administering the pharmaceutical liquid composition with an anti-epilepsy drug.

91. The method of treating a subject having the lysosomal storage disorder of any one or more of embodiment 83 to 90, where the administering or co-administering of the liquid pharmaceutical composition occurs once daily.

92. The method of treating a subject having the lysosomal storage disorder of any one or more of embodiment 83 to 90, where the administering or co-administering of the liquid pharmaceutical composition occurs two times daily.

93. The method of treating a subject having the lysosomal storage disorder of any one or more of embodiment 83 to 90, where the administering or co-administering of the liquid pharmaceutical composition occurs three times daily.

94. The method of treating a subject having the lysosomal storage disorder of any one or more of embodiment 83 to 90, where the administering or co-administering of the liquid pharmaceutical composition occurs four times daily.

### EXAMPLES

### Example 1

Formation of a liquid oral gemfibrozil solution was attempted by crushing gemfibrozil tablets from sachets and combining the contents with methanol. An assay preparation was performed by pooling together gemfibrozil sachets as follows: 5×30 mg sachets; 3×60 mg sachets; or 2×120 mg sachets. The sachets were pooled in a glass beaker for each of the doses, mixed with a metal micro-spatula, 77.2-79.2 mg of gemfibrozil was weighed into a volumetric flask to which 40 mL of methanol was added, and samples were placed on an orbital shaker for 30 minutes at 200 rpm. At this stage clumping of the material was not noticed. The samples were then brought to volume and inverted 15 times to ensure complete mixing of the 1.0 mg/mL stock solution that was formed. Dilutions were made to form working samples having a concentration of 0.2 mg/ml.

Content uniformity preparations were prepared from single sachets of each dose strength. The sachet contents were dispensed through a glass funnel into a volumetric flask, followed by rinsing of both the sachet and funnel with methanol. Upon addition of methanol to the flask clumping of the gemfibrozil material occurred. This clumping was most prominent with the 120-mg dose. Samples were placed on an orbital shaker for 30 minutes at 200 rpm, but the agitation did not disperse the cluster of clumped material. Sample preparation was completed as done in the assay preparation. The 120-mg dose had only a small amount of loose material present in the bottom while the rest was one large bulbous clump. The 120-mg dose had the higher percent recovery and smaller acceptance value (AV) value. However, the agglomerated material may trap some gemfibrozil within and prevent its full dissolution. This phenomenon varied between samples and dosages as it depends on how the powder is dispersed in the flask prior to methanol addition. Due to the amount of material required and the lack of unopened reference material, three vials were used for the analysis.

During the review of the assay work no lab error was observed. A check of the % w/w generated (reported as 99.7% label claim with an % RSD of 0.6%) from the blend uniformity data shows that the feasibility BMR was using a value that was 3.23% higher to calculate the target fill weight (the blend uniformity shows a % w/w of 63.77% but the feasibility uses a value of 67%). All the target fill weights documented in the batch manufacturing record (BMR) are between 3-10 mg of the target weight if the correct % w/w is used. The target weights and the actual weights that were required are illustrated below in Table 1.

TABLE 1

| | | BMR Target | Required Target | Difference |
|---|---|---|---|---|
| Target | | Weight (mg) - | weight (mg) - based | (actual/ |
| Dose | | based on 67% | on 63.77% w/w | required) * |
| Strength | | w/w | (from BU analysis) | 100)) |
| 30 | mg | 44.8 | 47.0 | 95.3% |
| 60 | mg | 89.6 | 94.1 | 95.2% |
| 120 | mg | 178.1 | 188.2 | 94.6% |

If the low assay for the 30-mg sachets is adjusted for the actual fill of gemfibrozil (95.3% of 30 mg=28.59 mg), the recovery result would be (30 mg*91.1/100)=27.33 mg (actual mg of gemfibrozil quantified during the assay). The percent of the actual amount (27.3 mg) relative to the actual fill (28.59 mg) is 95.6%. Following the same approach, the actual amount relative to the actual fill is 97.8% and 100.5% for the 60-mg sachet and 120-mg sachet, respectively. The low target fill weight (approx. 95% of required) would not have an impact on the low assay results reported at least because the method of sample preparation requires a pool of sachets to be made which is then used to weigh out the 78 mg amount of gemfibrozil sample for testing.

It was also noted that as the content uniformity requires one whole sachet for each preparation. Rinsing of the sachets to ensure that all the contents were removed was not performed during the assay preparation/pooled method due to the difference in preparation. As such there is the potential that gemfibrozil is sticking to the inside of the sachets. The analyst did confirm though that the pooled sachets are thoroughly scraped. Notably, the same size sachets are being used for all three dose strengths. Therefore, a greater internal surface area to amount of crushed tablet ratio is present for the 30-mg dose sachets than for the higher dose sachets; this ratio decreases in order of increasing dosage (and amount filled into the sachet). Accordingly, losses on the internal surface of the sachet would be greater for the 30-mg dose than for the 60-mg or 120-mg doses.

A review of the analytical method has shown that the final working concentration for the content uniformity samples is 0.96 mg/mL with a stock concentration of 1.2 mg/ml. The working sample concentration is higher than the standards and upon review of the validation of the method is also outside of the linear range. The stock concentration is also higher than the concentration at which gemfibrozil is known to be soluble in the diluents used; therefore, there is no certainty in the data generated.

To determine if the incorrect concentration of the content uniformity sample is the cause of the results and AV values seen, it is proposed that a selection of the stock solutions (at 1.2 mg/mL nominal concentration) for the 30-mg dose strength are diluted using 1.5 mL in 10.0 mL volumetric flask. This is for information purposes only because the solutions are outside of their stability window. For each even numbered content uniformity solution (2, 4, 6, 8 and 10) 1.5 mL was pipetted into a 10.0 mL volumetric flask and made to volume using the assay diluent.

Due to a suspected issue with the gemfibrozil adhering to the sachets, additional testing using the raw material and the blend intermediate sample was proposed. Assay testing was performed in unused crushed tablet: 78.41 mg±1 mg of crushed tablets in a 50 ml volumetric flask (based on the 63.7% w/w result from blend uniformity testing above). Sample were prepared as stated above, in triplicate. Content uniformity testing was performed on unused crushed tablets: 47 mg±1 mg of crushed tablets in 25 mL volumetric flask (based on 63.77% w/w result from blend uniformity testing above). Sample were prepared as stated above, in triplicate, for the 30-mg sachets with the final dilution step of 1.5 ml in 10 ml. An alternate content uniformity testing was performed on crushed tablets: 47 mg±1 mg of crushed tablets in 100 ml volumetric flask with the addition of 80 mL of methanol, which was inverted 15 times and further diluted using 7.0 mL into a 10 mL volumetric flask. A nominal concentration of 0.21 mg/mL was used to correct for concentration differences in the equation used for quantification.

For the assay, five sachets at the 30-mg dose were prepared by transferring 47 mg of crushed tablet into five separate sachets. Each was emptied into the same glass vial, the internal surface of the sachets was scraped, and the contents mixed with the spatula. From this, 78.41 mg of crushed tablet was accurately weighted and put into a 50 ml volumetric flask. All sachets were rinsed with methanol and transferred to a 10 ml volumetric flask, brought to volume with methanol, and mixed. 3.0 ml was diluted in a 10 ml flask and brought to volume with water and acetic acid (99/1 v/v). The samples were injected and quantified against freshly prepared standard at 0.2 mg/ml.

The re-dilution of the original stock solutions (prepared at 1.2 mg/ml) to within the established linear range improved the recovery of gemfibrozil by 2-3%.

Regarding the combining of the five 30-mg sachets, there was insufficient material left behind to make the third sample as it contained only 50 mg instead of 78.4 mg suggesting material is left behind in the sachet following emptying. To determine the percent amount left behind with the sachets, each was analyzed by rinsed with 1 ml of methanol into a 10 ml flask and further diluted as described. Table 2 below shows the assay value in mg that was found to be remaining in the sachets.

TABLE 2

| Sachet No | Wt. of Blend | Area | Assay % w/w | Rinse Assay (mg) |
|---|---|---|---|---|
| 1 | 47.85 | 208980 | 3.807 | 1.82 |
| 2 | 47.34 | 297107 | 5.412 | 2.56 |
| 3 | 47.30 | 168062 | 3.061 | 1.45 |
| 4 | 47.82 | 199500 | 3.634 | 1.74 |
| 5 | 46.93 | 193726 | 3.529 | 1.66 |
| Total | 237.24 | N/A | 19.44 | 9.23 |
| Mean | 47.45 | NA | 3.889 | N/A |
| % RSD | 0.82 | N/A | 23.0 | N/A |

The total amount of the blended sachets was 237.24 mg. Also, the total amount of blend weighed for the triplicate assay replicates was 207.29 mg. The difference between these is 29.96 mg. The total amount of gemfibrozil determined from the rinse assay of the empty sachets is 9.23 mg. Assuming a % w/w of blend from blend uniformity testing means 9.23 mg/0.6377=14.47 mg. Given the above, the amount lost during preparation is equal to 29.96 mg−14.47 mg=15.49 mg. This demonstrates that the process of emptying the sachets into a beaker to form a composite of crushed tablet leaves powder on the inside surface of the sachet, which would be difficult to remove without rinsing into the volumetric flask.

Table 3 below illustrates the assay data of just the crushed tablets, with no interference from the sachets. This demonstrates that the assay is approximately 3% higher when the sachets are not involved in the preparation.

TABLE 3

| Replicate No | % w/w | % label claim | Area |
|---|---|---|---|
| 1 | 62.374 | 97.8 | 454936 |
| 2 | 62.189 | 97.5 | 453039 |
| 3 | 62.383 | 97.8 | 452487 |
| Mean | 62.316 | 97.7 | N/A |
| % RSD | 0.2 | 0.2 | N/A |

Data from the “additional testing” of the content uniformity is illustrated below in Table 4.

TABLE 4

| Replicate | Dilution | % Label claim | Dilution | % Label claim |
|---|---|---|---|---|
| 1 | 25 mL | 94.3 | 100 mL | 98.5 |
| 2 | then 1.5 | 97.6 | then | 96.7 |
| 3 | in 10 mL | 98.4 | 7 mL | 97.2 |
| 4 | | 98.0 | in 10 mL | 97.8 |
| 5 | | 99.4 | | 97.7 |
| 6 | | 100.1 | | 98.9 |
| 7 | | 97.3 | | 98.7 |
| 8 | | 98.6 | | 99.0 |
| 9 | | 98.2 | | 98.4 |
| 10 | | 97.9 | | 98.8 |
| Mean | NA | 98.0 | NA | 98.2 |
| % RSD | NA | 1.6 | NA | 0.8 |
| AV | NA | 3.8 | NA | 2.2 |

The two approaches shown in Table 3 and Table 4 demonstrate similar results; however, the second set of preparations showed a lower % RSD and slightly higher mean result. These results are significantly higher than the original content uniformity results; however, they have been diluted to within the linear range and do not incorporate the sachet in the sample preparation. The most likely loss of API/crushed tablet from the original results are due to high sample concentrations (shown by the peak areas which have been tabulated) being outside the linear range and loss or incomplete transfer of material from the sachet. Therefore, a sample preparation technique for assay and content uniformity that adds the sachet to the flask for complete dissolution would be preferred, providing the sachets do not show any interfering peaks and specificity. However, even with sample weight and no influence from the sachets, the results are all lower than 100%.

Re-dilution of the content uniformity samples to bring them into the linear range improves the assay result, but not to within the intended specification. The most likely cause for this is the loss of powder/crushed gemfibrozil tablet during preparation. This could be either from dust losses when opening the sachet or incomplete transfer during the rinse step, especially if the tear-slot has been used. Preparation of the blend without the sachet confirms >97%.

Losses during sample preparation appear to be exaggerated for the low dose as more sachets are used in preparation, therefore assay results obtained for the 30-mg dose are lower than for the 60-mg dose and 120-mg dose. Preparation of a blend without the sachets confirms acceptable data >97%. Even though the results for all doses are within specification, they are on the lower side of specification; therefore, results obtained at T=0 for all doses will be invalidated.

### Example 3

Solubility testing for gemfibrozil indicated that gemfibrozil was soluble in a number of solvents and/or cosolvents. Two doses of gemfibrozil were considered for each solvent/cosolvent. A low dose gemfibrozil preparation: 60 mg gemfibrozil in 5 ml of solvent/cosolvent (12 mg/ml dose), and a high dose gemfibrozil preparation: 160 mg gemfibrozil in 5 mL of solvent/cosolvent (32 mg/ml dose). Table 5 below illustrates that the first six solvent/cosolvents achieved gemfibrozil solubilization at both the 12 mg/ml dose and the 32 mg/ml dose. The solvent/cosolvents included the sodium carbonate/bicarbonate buffer having a pH of 10.5, polyethylene glycol (PEG) 400, glyceryl monolinoleate, propylene glycol dicaprylate/dicaprate, propylene glycol, and glyceryl monooleate.

TABLE 5

| | Gemfibrozil Solubility |
|---|---|
| pH 10.5 Sodium carbonate/bicarbonate buffer | Soluble at 12 mg/ml and 32 mg/ml |
| Polyethylene glycol 400 | Soluble at 12 mg/ml and 32 mg/ml |
| Glyceryl monolinoleate | Soluble at 12 mg/ml and 32 mg/ml |
| Propylene glycol dicaprylate/dicaprate | Soluble at 12 mg/ml and 32 mg/ml |
| Propylene glycol | Soluble at 12 mg/ml and 32 mg/ml |
| Glyceryl monooleate | Soluble at 12 mg/ml and 32 mg/ml |
| pH 4.5 citrate buffer and TPGS | Soluble at 12 mg/ml |
| pH 4.5 Citrate buffer and Solutol HS15 | Not soluble |
| pH 4.5 Citrate buffer and Poloxamer P188 | Not soluble |
| Soybean oil | Not soluble |
| Glycerol and Polysorbate 80 | Not soluble |

The citrate buffer having a pH of 4.5 with tocopheryl polyethylene glycol succinate (TPGS) achieved gemfibrozil solubilization only the 12 mg/ml dose. The remainder of the solvents/cosolvents were not able to solubilize gemfibrozil at either the low dose or the high dose concentration.

### Example 4

Based on the solubility of gemfibrozil in at least PEG 400, propylene glycol, and a citrate buffer having a pH of 4.5, an excipient compatibility study was conducted using the low dose gemfibrozil as shown in Table 6 below. Three different prototype liquid formulation (P1, P2, and P3) were evaluated for gemfibrozil solubility.

TABLE 6

% w/w Composition

| | Prototype P1 | Prototype P2 | Prototype P3 |
|---|---|---|---|
| pH 4.6 citrate | 79.36 | 79.36 | 79.38 |
| TPGS | 8.82 | 8.82 | 8.82 |
| PEG 400 | 10.00 | — | 10.00 |
| Propylene glycol | — | 10.00 | — |
| Sodium saccharin | 0.10 | 0.10 | 0.10 |
| Methyl paraben | 0.20 | 0.20 | — |
| Ethyl paraben | 0.02 | 0.02 | — |
| Sodium benzoate | — | — | 0.20 |
| Cherry flavour | 0.30 | 0.30 | 0.30 |
| Gemfibrozil | 1.20 | 1.20 | 1.20 |
| Total | 100.00% | 100.00% | 100.00% |

The three different prototype liquid formulation (P1, P2, and P3) were evaluated for gemfibrozil chemical stability, and the growth of any gemfibrozil-related substances for up to 28 days of storage at either 25° C. or 50° C. There was no significant decrease in the chemical stability of gemfibrozil or growth of gemfibrozil-related substances up to 28 days of storage at either temperature in any of the prototype liquid formulations.

### Example 5

A concentrated stock solution of gemfibrozil was prepared in PEG 400. Gemfibrozil was evaluated for its solubility in PEG 400 over concentration range to determine an upper concentration that could be used to prepare a stock solution of gemfibrozil in liquid form. Seven different aliquots of gemfibrozil were evaluated as shown in Table 7 below. On the day of preparation gemfibrozil was dissolved in the PEG 400 at the indicated concentration. All samples were clear upon mixing. Following mixing, samples were stored standing upright at room temperature overnight. Upon standing overnight, Samples #1-6 remained in solution; however, Sample #7 (32.0% w/w gemfibrozil) precipitated out of solution. The solubility limit of gemfibrozil was determined to be 21.3% w/w in PEG 400.

TABLE 7

| Sample # = API Aliquot # | | 1 | 2 | 3 | 4 | 5 | 6 | 7 |
|---|---|---|---|---|---|---|---|---|
| Required [API] in PEG to achieve | (% w/w) | 8.0 | 9.1 | 10.7 | 12.8 | 16.0 | 21.3 | 32.0 |
| upper dose in final product | (mg/mL) | 80.0 | 91.4 | 106.7 | 128.0 | 160.0 | 213.3 | 320.0 |

### Example 6

The physical stability of a low dose gemfibrozil (3.2% weight/volume) in a citrate buffer having a pH 4.5 was evaluated over a range of PEG 400 concentrations, both in the presence and absence of TPGS. The total concentration of PEG 400 ranged from 46.8% weight/volume to 11.8% weight/volume in the final formulation. The PEG 400 concentrate from Sample #6 in Example 5 was used to prepare the formulations, with additional PEG 400 being added as necessary. Following preparation, samples were stored at either 15° C. or 18.5-25.0° C. for 64 hours.

Table 8 and Table 9 below illustrate the composition of the formulation with varying amounts of PEG 400 in either the absence of TPGS (Table 4; PO1-PO8) or presence of TPGS (Table 5; PT1-PT8).

TABLE 8

| | Composition (% w/v of final formulation) | | | | | | | |
|---|---|---|---|---|---|---|---|---|
| Formulation Code | PO1 | PO2 | PO3 | PO4 | PO5 | PO6 | PO7 | PO8 |
| Target API (T)² | 3.2 | 3.2 | 3.2 | 3.2 | 3.2 | 3.2 | 3.2 | 3.2 |
| [API] in PEG Concentrate (A [symbol] )² | 21.3 | 21.3 | 21.3 | 21.3 | 21.3 | 21.3 | 21.3 | 21.3 |
| PEG from PEG concentrate (P [symbol] )² | 11.8 | 11.8 | 11.8 | 11.8 | 11.8 | 11.8 | 11.8 | 11.8 |
| Total PEG In formulation | 46.8 | 41.8 | 36.8 | 31.8 | 26.8 | 21.8 | 16.8 | 11.8 |
| PEG Concentrate in final formulation (C [symbol] )² | 15.0 | 15.0 | 15.0 | 15.0 | 15.0 | 15.0 | 15.0 | 15.0 |
| Additional PEG 400 in final formulation | 35.0 | 30.0 | 25.0 | 20.0 | 15.0 | 10.0 | 5.0 | 0.0 |
| pH 4.5 buffer in formulation | q.s. | q.s. | q.s. | q.s. | q.s. | q.s. | q.s. | q.s. |
| Total | 100.0 | 100.0 | 100.0 | 100.0 | 100.0 | 100.0 | 100.0 | 100.0 |

[symbol]

indicates data missing or illegible when filed

TABLE 9

| | Composition (% w/v of final formulation) | | | | | | | |
|---|---|---|---|---|---|---|---|---|
| Formulation Code | PT1 | PT2 | PT3 | PT4 | PT5 | PT6 | PT7 | PT8 |
| Target API (T)² | 3.2 | 3.2 | 3.2 | 3.2 | 3.2 | 3.2 | 3.2 | 3.2 |
| [API] in PEG Concentrate (A [symbol] )² | 21.3 | 21.3 | 21.3 | 21.3 | 21.3 | 21.3 | 21.3 | 21.3 |
| PEG from PEG concentrate (P [symbol] )² | 11.8 | 11.8 | 11.8 | 11.8 | 11.8 | 11.8 | 11.8 | 11.8 |
| Total PEG In formulation | 46.8 | 41.8 | 36.8 | 31.8 | 26.8 | 21.8 | 16.8 | 11.8 |
| TPGS in formulation | 6.0 | 6.0 | 6.0 | 6.0 | 6.0 | 6.0 | 6.0 | 6.0 |
| 20% TPGS Concentrate in pH 4.5 Buffer | 30.0 | 30.0 | 30.0 | 30.0 | 30.0 | 30.0 | 30.0 | 30.0 |
| PEG Concentrate in final formulation (C [symbol] )² | 15.0 | 15.0 | 15.0 | 15.0 | 15.0 | 15.0 | 15.0 | 15.0 |
| Additional PEG 400 in final formulation | 35.0 | 30.0 | 25.0 | 20.0 | 15.0 | 10.0 | 5.0 | 0.0 |
| pH 4.5 buffer in formulation | q.s. | q.s. | q.s. | q.s. | q.s. | q.s. | q.s. | q.s. |
| Total | 100.0 | 100.0 | 100.0 | 100.0 | 100.0 | 100.0 | 100.0 | 100.0 |

[symbol]

indicates data missing or illegible when filed

Table 10 below illustrates that in the absence of TPGS none of the gemfibrozil formulations were clear upon mixing. Following storage for 64 hours at the indicated temperatures, all samples were cloudy with a visible precipitate. Notably, there phase separation along with a precipitate following storage in the P08 samples at both storage temperatures.

TABLE 10

| Sample Label | PO1 | PO2 | PO3 | PO4 | PO5 | PO6 | PO7 | PO8 |
|---|---|---|---|---|---|---|---|---|
| Appearance immediately | Cloudy with | Cloudy with | Cloudy with | Cloudy with | Cloudy with | Cloudy with | Cloudy with | Cloudy with |
| after Mixing | precipitate | precipitate | precipitate | precipitate | precipitate | precipitate | precipitate | precipitate |
| Appearance after 64 | Cloudy with | Cloudy with | Cloudy with | Cloudy with | Cloudy with | Cloudy with | Cloudy with | Two phases |
| hours at ~15° C. | precipitate | precipitate | precipitate | precipitate | precipitate | precipitate | precipitate | with precipitate |
| Appearance after 64 | Cloudy with | Cloudy with | Cloudy with | Cloudy with | Cloudy with | Cloudy with | Cloudy with | Two phases |
| hours at 18.5-25.0° C. | precipitate | precipitate | precipitate | precipitate | precipitate | precipitate | precipitate | with precipitate |

Table 11 below illustrate that in the presence of TPGS some cloudiness was observed across all formulations (PT1-PT8). There was no precipitate observed immediately after mixing in samples PT1-PT6; however, a precipitate was observed in the PT7 and PT8 formulations immediately after mixing. Following storage for 64 hours at the indicated temperatures, all samples were cloudy with a visible precipitate at all amounts of PEG 400. Notably, there phase separation along with a precipitate following storage in the PT5-PT8 samples at 18.5-25.0° C.

TABLE 11

| Sample Label | PT1 | PT2 | PT3 | PT4 | PT5 | PT6 | PT7 | PT8 |
|---|---|---|---|---|---|---|---|---|
| Appearance | Cloudy, No | Cloudy, No | Cloudy, No | Cloudy, No | Cloudy, No | Cloudy, No | Cloudy with | Cloudy with |
| immediately | precipitate | precipitate | precipitate | precipitate | precipitate | precipitate | precipitate | precipitate |
| after Mixing | | | | | | | | |
| Appearance | Cloudy with | Cloudy with | Cloudy with | Cloudy with | Cloudy with | Cloudy with | Cloudy with | Cloudy with |
| after 64 hours | precipitate | precipitate | precipitate | precipitate | precipitate | precipitate | precipitate | precipitate |
| at ~15° C. | | | | | | | | |
| Appearance | Cloudy with | Cloudy with | Cloudy with | Cloudy with | Phase separation | Phase separation | Phase separation | Phase separation |
| after 64 hours | precipitate | precipitate | precipitate | precipitate | with precipitate | with precipitate | with precipitate | with precipitate |
| at 18.5- | | | | | | | | |
| 25.0° C. | | | | | | | | |

Based on the data of Example 6 it was determined that PEG 400 is not able to physically stabilize gemfibrozil in citrate buffer solutions having a pH 4.5 even though PEG 400 is a good solvent for gemfibrozil.

### Example 7

Initial taste evaluation was performed using a carbonate/bicarbonate buffer having a pH 10.5 with bitrex (25 ppb) and without bitrex. The composition of the formulation used for the taste evaluation is illustrated below in Table 12. The concentration of both the cherry flavor and the sweetener (sodium saccharin) were varied within each of the 5 different formulations.

TABLE 12

| | % w/v | | | | | % w/w |
|---|---|---|---|---|---|---|
| | Composition | | | | | Composition |
| | Formulation Reference | | | | | |
| | F [symbol] 1 | F [symbol] 2 | F [symbol] 3 | F [symbol] 4 | F [symbol] 5 | F [symbol] 1 |
| | Batch reference = POL006/. . . | | | | | |
| | 40F [symbol] 1 | 44F [symbol] 2 | 46F [symbol] 3 | 50F [symbol] 4 | 53F [symbol] 5 | 60F [symbol] 1 |
| pH 10.5 Carbonate/Bicarbonate Buffer, | 94.00 | 94.00 | 94.00 | 94.00 | 94.00 | 93.63 |
| Portion 1 | | | | | | |
| Cherry flavour [symbol] | 0.10 | 0.03 | 0.30 | 0.30 | 0.03 | 0.10 |
| Sodium saccharin | 0.05 | 0.02 | 0.20 | 0.02 | 0.20 | 0.05 |
| 0.1M HCl or 0.1M NaOH | nil | nil | nil | nil | nil | nil |
| pH 10.5 Carbonate/Bicarbonate Buffer, | 7.58 | 6.94 | 10.37 | 6.87 | 7.12 | 6.22 |
| Portion 2 | | | | | | |
| Total | 101.73 | 100.99 | 104.87 | 101.19 | 101.35 | 100.00 |
| pH | 10.38 | 10.45 | 10.24 | 10.28 | 10.44 | 10.44 |
| Density (g/mL) | 1.01 | 1.01 | 1.01 | 1.01 | 1.01 | 1.01 |

[symbol]

indicates data missing or illegible when filed

The taste panel evaluation of the five formulations in Table 12 is summarized below in Table 13. The best formulation in terms of taste acceptability was the F_{Carb1} formulation having a sodium saccharin concentration of 0.05% and cherry flavor concentration of 0.1%, both concentrations being at an intermediate level for each excipient. The next best formulations in terms of taste acceptability were the F_{Carb3} and F_{Carb5} formulations. The different concentrations of cherry in these two formulations did not impact taste acceptability. Neither formulation with the lowest concentration of sodium saccharin (F_{Carb2} and F_{Carb4}) were found to be taste acceptable to any tasters with or without bitrex spiking.

TABLE 13

| | Formulation Reference | | | | | | | | | |
|---|---|---|---|---|---|---|---|---|---|---|
| | F_{CARB}1 | | F_{CARB}2 | | F_{CARB}3 | | F_{CARB}4 | | F_{CARB}5 | |
| | Batch reference = POL006/. . . | | | | | | | | | |
| | (40F_{CARB}1) | | (44F_{CARB}2) | | (46F_{CARB}3) | | (50F_{CARB}4) | | (53F_{CARB}5) | |
| [Sodium saccharin] | 0.05 | | 0.020 | | 0.20 | | 0.02 | | 0.20 | |
| (% w/w) | | | | | | | | | | |
| [Cherry Flavour] | 0.10 | | 0.03 | | 0.30 | | 0.30 | | 0.03 | |
| (% w/w) | | | | | | | | | | |
| Bitrex Spiked? | Yes | No | Yes | No | Yes | No | Yes | No | Yes | No |
| Number of tasters that | 3 | 2 | None | None | 1 | 1 | None | None | 2 | 1 |
| found the taste acceptable | | | | | | | | | | |
| (out of 3) | | | | | | | | | | |

E-Tongue analysis of gemfibrozil prototype formulations was also performed at both low-dose gemfibrozil and high-dose gemfibrozil, and compared with placebo controls with and without bitrex. The low-dose gemfibrozil formulation was concluded to be similar to placebo controls, and the high-dose gemfibrozil was concluded to be different to the two placebo controls, but not necessarily unacceptable in terms of taste. Both gemfibrozil formulations were prepared according to Table 14 below.

### Example 8

The stability of gemfibrozil in a carbonate buffer having a pH of 10.5 (+/−0.5) was evaluated upon mixing, at 14 days after mixing, and at 28 days after mixing under storage conditions of approximately 25° C. and 40° C. Table 14 below illustrate the composition of the liquid pharmaceutical composition used with both a 12 mg/ml gemfibrozil formulation (F_{CARB}LD1) and a 32 mg/ml gemfibrozil formulation (F_{CARB}HD1).

TABLE 14

| | % w/v Composition | | % w/w Composition | |
|---|---|---|---|---|
| Formulation Reference | F_{CARB}HD1 | F_{CARB}LD1 | F_{CARB}HD1 | F_{CARB}LD1 |
| Batch reference = POL006/ . . . | 64FHD1 | 70FLD1 | 64FHD1 | 70FLD1 |
| pH 10.5 Carbonate/Bicarbonate | 94.00% | 94.00% | 93.30% | 93.17% |
| Buffer, Portion 1 | | | | |
| Cherry flavour^{A, Page7} | 0.10% | 0.10% | 0.10% | 0.10% |
| Sodium saccharin | 0.05% | 0.05% | 0.05% | 0.05% |
| Gemfibrozil | 3.20% | 1.20% | 3.18% | 1.19% |
| 1M NaOH | 5.70% | 5.33% | 5.65% | 5.29% |
| 0.1M NaOH | 2.36% | — | 2.34% | — |
| pH 10.5 Carbonate/Bicarbonate | 5.05% | 1.13% | 5.01% | 1.12% |
| Buffer, Portion 2 | | | | |
| Total | 110.46% | 101.82% | 109.64% | 100.91% |
| Density = mass of solution/1000 mL | 1.007 | 1.009 | | |
| flask volume (g/mL) | | | | |
| pH (i) before adjustment, | (i) 9.01 | (i) 9.48 | | |
| (ii) after adjustment | (i) 10.48 | (ii) 10.49 | | |

Table 15 below illustrates the stability and pH of the low dose (12 mg/ml) and high dose (32 mg/ml) liquid pharmaceutical composition comprising gemfibrozil. All formulations are clear upon mixing with a slightly yellow color. Precipitate was only observed in the PT7 and PT8 formulations. Following either 14 days of storage or 28 days of storage at either 25° C. and 40° C. did not significantly alter the appearance of the formulation. The density remained unchanged throughout storage at either temperature. The pH was unchanged at 14 days of storage at both 25° C. and 40° C.; however, the pH decreased slightly at 28 days of storage at both 25° C. and 40° C.

TABLE 15

| | | | | Density | |
|---|---|---|---|---|---|
| Sample | Time Point | Condition | Appearance | (g/cm³) | pH |
| Low dose 60 | During preparation | | N/A | 1.01 | 10.49 |
| mg/5 mL | Initial | N/A | A clear, | 1.01 | 10.35 |
| | | | slightly | | |
| | | | yellow liquid | | |
| | | | with the | | |
| | | | aroma of | | |
| | | | cherry. Free | | |
| | | | from | | |
| | | | particulates* | | |
| | 14 days | 25° C. | As initial⁵ | 1.01 | 10.38 |
| | | 40° C. | As initial⁵ | 1.01 | 10.35 |
| | 28 days | 25° C. | As initial | 1.01 | 10.18 |
| | | 40° C. | As initial | 1.01 | 10.17 |
| High dose | During preparation | | N/A | 1.01 | 10.48 |
| 160 mg/5 mL | Initial | N/A | A clear, | 1.01 | 10.31 |
| | | | slightly | | |
| | | | yellow liquid | | |
| | | | with the | | |
| | | | aroma of | | |
| | | | cherry. Free | | |
| | | | from | | |
| | | | particulates* | | |
| | 14 days | 25° C. | As initial⁵ | 1.01 | 10.31 |
| | | 40° C. | As initial⁵ | 1.01 | 10.33 |
| | 28 days | 25° C. | As initial | 1.01 | 10.13 |
| | | 40° C. | As initial | 1.01 | 10.20 |

Table 16 below illustrates the stability the low dose (12 mg/ml) and high dose (32 mg/ml) liquid pharmaceutical compositions comprising gemfibrozil. No related substances were detected in either the low dose (12 mg/ml) or the high dose (32 mg/ml) gemfibrozil formulations at either storage time and temperature.

TABLE 16

| | Time | | | | Related |
|---|---|---|---|---|---|
| Sample | Point | Condition | Assay (%) | | Substances |
| Low dose | Initial | N/A | 100.0 | 100.1 | None detected |
| 60 mg/5 mL | | | 100.2 | (12.0 mg/mL) | |
| | 14 days | 25° C. | 100.6 | 100.5 | None detected |
| | | | 100.5 | (12.1 mg/mL) | |
| | | 40° C. | 101.4 | 101.4 | None detected |
| | | | 101.3 | (12.2 mg/mL) | |
| | 28 days | 25° C. | 99.8 | 99.9 | None detected |
| | | | 100.0 | (12.0 mg/mL) | |
| | | 40° C. | 99.9 | 99.9 | None detected |
| | | | 99.9 | (12.0 mg/mL) | |
| High dose | Initial | N/A | 100.4 | 100.5 | None detected |
| 160 mg/5 | | | 100.7 | (32.2 mg/mL) | |
| mL | 14 days | 25° C. | 102.2 | 101.3 | None detected |
| | | | 100.4 | (32.4 mg/mL) | |
| | | 40° C. | 101.3 | 101.0 | None detected |
| | | | 100.8 | (32.3 mg/mL) | |
| | 28 days | 25° C. | 100.7 | 100.7 | None detected |
| | | | 100.7 | (32.2 mg/mL) | |
| | | 40° C. | 100.8 | 100.6 | None detected |
| | | | 100.5 | (32.2 mg/mL) | |

The liquid pharmaceutical compositions comprising gemfibrozil used in this example were prepared on a 1-liter scale. Heating the formulation to approximately 50° C. during stirring helped to achieve full solubilization of the high dose gemfibrozil sample (32 mg/ml). HPLC data demonstrated that chemical degradation of gemfibrozil occurred at this elevated temperature. These conditions did result in some evaporation (approximately 8%). Table 17 below illustrates the time, stirring, and temperature variations used to dissolve the gemfibrozil in the high-dose formulation.

TABLE 17

| | Time to dissolve during mixing [mixing speed in | |
|---|---|---|
| | brackets] (ambient temperature unless stated) | |
| Formulation Reference | F_{CARB}HD1 | F_{CARB}LD1 |
| Batch reference | POL006/64FHD1 | POL006/70FLD1 |
| Sodium saccharin | 11 s | 10 s |
| | [250 RPM] | [250 RPM] |
| Gemfibrozil API | Complete after: | 263 min |
| | (i) 15 min [280 RPM] @ ambient | [250 RPM] |
| | (ii) 187 min [280 RPM] @ 47-50° C. | |
| | (iii) 1231 min [230 RPM] at ambient | |
| | (iv) 30 min [245 rpm] at ~50° C. | |

### Example 9

The preparation and evaluation of 8 different liquid pharmaceutical composition comprising gemfibrozil was conducted. The four formulations were prepared in a carbonate buffer having a pH of 10.5 (+/−0.5), the first having a high dose of gemfibrozil (30 mg/ml; Sample F_{CARB30}), the second having a lower dose of gemfibrozil (15 mg/ml; Sample F_{CARB15}), the third having the highest dose of gemfibrozil (F_{CARBHD}; 32 mg/ml), and the fourth having the lowest dose of gemfibrozil (F_{CARBLD}; 12 mg/ml). No preservatives or other cosolvents/surfactants were included in the F_{CARB15} Or F_{CARB30} formulations. Another two formulations were prepared in a citrate buffer having a pH of 4.5 (+/−0.5), the first having solutol HS15 as a cosolvent (Sample 4.5S) and the second having TPGS as a cosolvent (Sample 4.5T). Preservatives were included in the 4.5S and 4.5T formulations to minimize microbial growth; ethyl paraben and sodium methyl paraben, or sodium benzoate. An additional two formulations were prepared in a phosphate buffer having a pH of 6.8 (+/−0.5), the first having solutol HS15 as a cosolvent (Sample 6.8S) and the second having TPGS as a cosolvent (Sample 6.8T). Preservatives were included in the 6.8S and 6.8T formulations to minimize microbial growth; ethyl paraben and sodium methyl paraben, or sodium benzoate. Table 18 illustrates the components of the 6 different gemfibrozil liquid formulations referenced.

TABLE 18

| | Lead or Backup? | | | | | | | |
|---|---|---|---|---|---|---|---|---|
| | Lead Basis (multiple doses) | | | | Backup Formulations | | | |
| | Formulation Code # | | | | | | | |
| | F [symbol] | F [symbol] | F [symbol] | F [symbol] | 6.8S | 6.8T | 4. [symbol] S | 4. [symbol] T |
| | Aqueous buffer | | | | | | | |
| | pH 10.5 Na bicarbonate/carbonate | | | | pH 6.8 phosphate | | pH 4. [symbol] Citrate | |
| | Concentration of Ingredients (mg/mL) | | | | | | | |
| Gemfibrozil | 32.0 | 12.0 | 30.0 | 15.0 | 5.0 | 5.0 | [symbol] .0 | [symbol] .0 |
| TPGS (Vitamin E TPOS) | None | | | | | 120 | | 120 |
| HS15 (Kolliphor HS15) | | | | | 200 | | 200 | |
| SMP (Sodium Methyl | | | | | 2.2 | | | 2.2 |
| Paraben) | | | | | | | | |
| EP (Ethyl Paraben) | | | | | 0.2 | | | 0.2 |
| SB (Sodium Benzoate) | | | | | | 3.0 | 3.0 | |

| | |
|---|---|
| Sodium saccharin | 0.5 |
| Black Cherry 501027 A7 | 1.0 |

[symbol]

indicates data missing or illegible when filed

Preparations of the F_{CARB} formulations included mixing the carbonate buffer having pH 10.5 (sodium carbonate+sodium bicarbonate+water+1.0 M NaOH), sodium saccharin, and black cherry flavor at room temperature in a stainless-steel vessel until the solids dissolved. The solution was then heated to 65° C. and gemfibrozil was added and mixed at 65° C. until the solids dissolved. Unless as indicated for the F_{CARBLD} in which samples were prepared at ambient temperature. Samples were cooled to 20° C. to 25° C. while stirring, the pH was adjusted to 10.5+/−0.05 with NaOH and the sample was brought to volume (1000 ml in this case) with either water or carbonate buffer having pH 10.5.

Preparations of the 4.5S, 4.5T, 6.8S, and 6.8T formulations included mixing either the citrate buffer having pH 4.5 or the phosphate buffer having pH 6.8, with either HS15 or TPGS preheated to 50° C. in a stainless-steel vessel. The samples were then heated to 65° C. until the solids dissolved and then transferred to glass vials. Preservative, sodium saccharin, black cherry flavor, and gemfibrozil were added sequentially and mixed at 65° C. until each solid dissolved before adding the next. Samples were cooled to 20° C. to 25° C. while stirring, the pH was adjusted to 4.5+/−0.05 or 6.8+/−0.05 with NaOH or HCl accordingly and the sample was transferred back to a stainless-steel vessel and brought to volume (1000 ml in this case) with either water the corresponding buffer solution. The final concentration of gemfibrozil in each of these four formulations was only 5 mg/ml.

Table 19 below demonstrates the temperature, mixing speed and time to dissolve each of the components in solution for the F_{CARB30}, F_{CARB15}, F_{CARBHD}, and F_{CARBLD} formulations.

TABLE 19

| | Mixing parameters (in blue) for Batch Codes (in red) at gemfibrozil API concentrations (green in mg/mL) | | | | | | | | | | | |
|---|---|---|---|---|---|---|---|---|---|---|---|---|
| | Temperature (°C., mean of start | | | | | | | | | | | |
| | and end readings where recorded) | | | | Time to dissolve (min) | | | | Mixing speed (RPM) | | | |
| | 12 | 15 | 30 | 32 | 12 | 15 | 30 | 32 | 12 | 15 | 30 | 32 |
| Ingredient Added | F_{CARBLD}^{‡} | F_{CARB15} | F_{CARB30} | F_{CARBHD}^{‡} | F_{CARBLD}^{‡} | F_{CARB15} | F_{CARB30} | F_{CARBHD}^{‡} | F_{CARBLD}^{‡} | F_{CARB15} | F_{CARB30} | F_{CARBHD}^{‡} |
| Sodium saccharin | Amb* | Amb* | Amb* | Amb* | N/R* | 1.2 | 1.5 | N/R* | N/R* | 400 | 400 | N/R* |
| Black Cherry | Amb* | Amb* | Amb* | Amb* | 0.2 | 1.5 | 1.3 | 0.2 | 250 | 400 | 400 | 250 |
| Gemfibrozil API | Amb* | 65.2 | 65.2 | a) Amb* | 263 | 3.0 | 65.6 | a) 15 | 250 | 400 | 400 | a) 280 |
| | | | | b) 47-50 | | | | b) 187 | | | | b) 280 |
| | | | | c) Amb* | | | | c) 1231 | | | | c) 230 |
| | | | | d) 50 | | | | d) 30 | | | | d) 245 |

Sodium saccharin and cherry flavor were readily dissolved in all formulations. The dissolution time of gemfibrozil was much shorter in the F_{CARB15} samples than in the F_{CARB30} samples. The dissolution time of gemfibrozil was also much shorter in the F_{CARB15} sample (15 mg/ml gemfibrozil) mixed at 65° C. than in the F_{CARBLC} sample (12 mg/ml gemfibrozil) mixed at ambient temperature. Increasing the temperature to 65° C. decreased the dissolution time of the gemfibrozil.

Table 20 below indicates that evaporation of water occurs at the elevated mixing temperatures compared with ambient. Water lost by evaporation ranged from 8%-15% in the samples prepared at 65° C. compared with a less than 1% loss of water in the sample prepared at ambient temperature. The pH also decreased in all four of these formulations during preparation and needed to be adjusted after manufacture. The pH in the F_{CARB30} and F_{CARB15} formulations was decreased by 2.7 and 1.7 pH units, respectively, in these samples.

TABLE 20

| | F_{CARBLD1} | | | F_{CARBD1} |
|---|---|---|---|---|
| Formulation Code | (TR-17-261) | F_{CARB15} | F_{CARB30} | (TR-17-261) |
| Water lost by Evaporation | 0.92% | 8.15% | 12.62% | 9.76% |
| (as % w/w of materials added | | | | |
| Mass of 1M NaOH added (as % w/v) | 5.33% | 5.55% | 4.39% | 5.70% |
| 0.1M NaOH (as % w/v) | — | 7.29% | Not used | 2.36% |
| | pH measurements during and after manufacture | | | |
| pH before adjustment, | 9.48 | 8.73 | 7.877 | 9.01 |
| ph after adjustment | 10.49 | 10.46 | 10.50 | 10.48 |
| T = 0 testing | 10.35 | 10.61 | 10.54 | 10.31 |

Following preparation of these four samples, a precipitate was noted in only the F_{CARB30} sample both immediately after preparation and after greater than 2 days in storage at 15° C. The precipitate appeared as plate-like, long birefringent needle-like crystals. Physical instability was not evident after more than 2 days in storage in formulations prepared with 32 mg/ml gemfibrozil when the samples were stored at approximately 20° C.: there was no appearance of a precipitate. Physical instability was also not evident after more than 2 days in storage in formulations prepared with 15 mg/ml of gemfibrozil (F_{CARB15}).

There was no apparent change in the density of any of the four liquid pharmaceutical compositions; all densities were close to 1.0. The F_{CARB15} and F_{CARB30} samples also demonstrated acceptably low levels of microbes (absence of E. Coli, less than 10 cfu/ml of aerobic microbial count and yeast and mold count) immediately after preparation.

Table 21 below demonstrates the temperature, mixing speed and time to dissolve each of the components in solution for the 4.5S, 4.5T, 6.8S, and 6.8T formulations.

TABLE 21

| | Mixing parameters (in blue) for Batch Codes (in red) | | | | | | | | | | | |
|---|---|---|---|---|---|---|---|---|---|---|---|---|
| | Temperature (°C., mean of start | | | | | | | | | | | |
| | and end readings where recorded) | | | | Time to dissolve (mis) | | | | Mixing speed (RPM) | | | |
| Ingredient Added | 6.8S | 6.8T | 4.5S | 4.5T | 6.8S | 6.8T | 4.5S | 4.5T | 6.8S | 6.8T | 4.5S | 4.5T |
| TPGS | N/A* | 62.4 | N/A* | 63.8 | N/A* | 120 | N/A* | 190 | N/A* | 400 | N/A* | 160-2000 |
| HS15 | 63.1 | N/A* | 64.1 | N/A* | 8.5 | N/A* | 7.7 | N/A* | 260 | N/A* | 260 | N/A* |
| SB | N/A* | 63.3 | 64.7 | N/A* | N/A* | 3.9 | 5.9 | N/A* | N/A* | 260 | 260 | N/A* |
| EP | 65.2 | N/A* | N/A* | 65.0 | 1.7 | N/A* | N/A* | 2.6 | 260 | N/A* | N/A* | 160 |
| SMP | 65.2 | N/A* | N/A* | 64.8 | 3.5 | N/A* | N/A* | 4.4 | 260 | N/A* | N/A* | 160 |
| Sodium saccharin | 65.3 | 63.1 | 64.9 | 68.5 | 2.0 | 1.4 | 1.5 | 1.5 | 260 | 260 | 260 | 160 |
| Black Cherry | 62.6 | 63.1 | 65.0 | 63.9 | 2.3 | 2.0 | 2.3 | 5.3 | 260 | 260 | 260 | 700 |
| Gemfibrozil API | 61.0 | 63.2 | 64.2 | 61.4 | 11.3 | 15.3 | 12.6 | 14.4 | 260 | 150 | 260 | 700 |

Sodium saccharin and cherry flavor were readily dissolved in all formulations. The preservatives all dissolved in under 6 minutes with the pre-addition of surfactant (TPGS or HS15) and mixing at 65° C. The dissolution time of gemfibrozil was in the range of 11-15 minutes for all formulations.

The addition of TPGS in both 6.8T and 4.5T formulations resulted in the formation of gelatinous limps, which were scraped from the stirrer and back into the beaker. The stirrer speed was carefully set to ensure the surface was in continuous motion. The addition of HS15 into the 6.8S formulation resulted in foam formation due to the fast stirrer speed setting that caused aeration. The contents of the stainless-steel vessel were transferred to the glass vessel to observe the dissolution of the solids. Stirrer speeds were adjusted in the 4.5S formulation based on learnings from the 6.8S formulation preparation.

Table 22 below indicates that evaporation of water occurs at elevated mixing temperatures. Water lost by evaporation ranged from 6%-13%. The pH also varied slightly in each of the four formulations during preparation; however, this was within the 0.5 pH units acceptable per protocol. Adjustment of pH was performed after manufacture regardless.

TABLE 22

| Formulation Code | 6.8S | 6.8T | 4.5S | 4.5T |
|---|---|---|---|---|
| Water lost by Evaporation | 6.87% | 13.28% | 6.57% | 10.22% |
| (as % w/w of materials added) | | | | |
| 0.1M NaON | — | 8.18% | — | — |
| 1M HCl | — | — | 1.45% | — |
| 0.1M HCl | 8.34% | — | 11.37% | 12.88% |
| | pH measurements during and after manufacture | | | |
| pH before adjustment | 7.17 | 6.57 | 4.95 | 4.77 |
| pH after adjustment | 6.82 | 6.81 | 4.51 | 4.53 |
| T = 0 testing | 6.81 | 6.78 | 4.52 | 4.51 |

There was no apparent change in the density of any of these four formulations; all densities were close to 1.0. Low levels of microbes may have been present; however, this cannot be confirmed as micro testing was not required immediately after preparation.

Assay results of gemfibrozil and preservatives was determined using a gemfibrozil assay, which demonstrated that gemfibrozil ranged between 99.0% to 100.2% as shown in Table 23 below. The assay results of gemfibrozil are within 5% of nominal. HPLC chromatograms indicate that no related substances formed outside of the allowed limitations.

TABLE 23

| | Gemfibrozil Assay | | | SB Assay | | | SMP Assay | | | EP Assay | | |
|---|---|---|---|---|---|---|---|---|---|---|---|---|
| Formulation | (% of nominal) | | | (% of nominal) | | | (% of nominal) | | | (% of nominal) | | |
| Code | Rep 1 | Rep 2 | Mean | Rep 1 | Rep 2 | Mean | Rep 1 | Rep 2 | Mean | Rep 1 | Rep 2 | Mean |
| F_{CARB30} | 99.0 | 99.1 | 99.0 | N/A | N/A | N/A | N/A | N/A | N/A | N/A | N/A | N/A |
| F_{CARB15} | 100.2 | 100.1 | 100.2 | N/A | N/A | N/A | N/A | N/A | N/A | N/A | N/A | N/A |
| 6.8S | 98.6 | 98.8 | 98.7 | N/A | N/A | N/A | 97.0 | 97.0 | 97.0 | 97.5 | 97.4 | 97.5 |
| 6.8T | 96.1 | 96.1 | 96.1 | 97.3 | 98.0 | 97.7 | N/A | N/A | N/A | N/A | N/A | N/A |
| 4.5T | 97.3 | 97.5 | 97.4 | NA | N/A | N/A | 96.8 | 95.3 | 96.1 | 97.4 | 95.7 | 96.5 |
| 4.5S | 100.0 | 100.1 | 100.1 | 95.3 | 94.9 | 95.1 | N/A | N/A | N/A | N/A | N/A | V/A |

[symbol]

[symbol]

indicates data missing or illegible when filed

The assay results of gemfibrozil, and the preservatives (sodium benzoate (SB), sodium methyl paraben (SMP), and ethyl paraben (EP)) are within 5% of nominal. However, trends indicate that the 6.8S, 6.8T, and 4.5T formulations have the lowest API and preservative assay results. HPLC chromatograms indicate that there is a small peak at RRT 0.45 in the 6.8T formulation only of unknown origin.

### Example 10

The stability of gemfibrozil in a liquid pharmaceutical composition comprising gemfibrozil (15 mg/ml) in a carbonate buffer having a pH of 10.5 (+/−0.5), as well as the appearance, pH, density, microbial purity, and self-preserving ability of the liquid pharmaceutical composition comprising gemfibrozil, were evaluated at 1 month, 2 months, 3 months, 6 months, 9 months, 12 months, 18 months, and 24 months after preparation under storage conditions of approximately 2-8° C. or 25° C. with 60% relative humidity. The stability, appearance, pH, density, microbial purity, and self-preserving ability were also assessed for 1 month, 2 months, 3 months, and 6 months after preparation at 40° C. with 75% relative humidity. All liquid pharmaceutical composition samples were stored either upright or inverted for the indicated time period. No preservatives or other cosolvents/surfactants were included in these F_{CARB15} formulations.

Following preparation of the liquid pharmaceutical composition comprising gemfibrozil, the samples were colorless to pale yellow, free from precipitate, and had the aroma of cherry. Table 24 below illustrates that following storage at 2-8° C., after 1 month the samples were clear, colorless, and contained a crystalline precipitate; after 2 months the samples were clear, colorless, and free from crystalline precipitate; after 3 months the samples were clear, colorless, and contained a crystalline precipitate in the inverted sample but was free from precipitate in the upright sample; and after 6 months, 9 months, 12 months, 18 months, and 24 months the samples were clear, colorless, and free from crystalline precipitate. The crystalline precipitate that formed at 1 month and 3 months following storage was able to be reconstituted at each time point and testing was performed on these samples under ambient conditions.

TABLE 24

| Storage | | | Time Point | | |
|---|---|---|---|---|---|
| Condition | Specification | Method | (Months) | Result | |
| Initial | Colourless to | AM003 v10 | Initial | Pale yellow, clear solution, free from | |
| | pale yellow, clear | | (QPS15648) | precipitate, with the aroma of cherry⁵ | |
| | solution, free | | | | |
| | from precipitate, | | | | |
| | with the aroma of | | | | |
| | cherry | | | | |
| | | | | Inverted | Upright |
| 2-8° C. | Clear, colourless to | AM003 v10 | 1 Month | Clear, colourless | Clear, colourless |
| | pale yellow | | (QPS16081 - Upright, | solution containing | solution containing |
| | solution, free from | | QPS16082 - Inverted) | numerous particles of | numerous particles of |
| | crystalline | | | crystalline precipitate^{8,9} | crystalline precipitate^{8,9} |
| | precipitate.⁵ | AM003 v10 | 2 Months | Clear, colourless | Clear, colourless |
| | | | (QPS16357 - Upright, | solution free from | solution free from |
| | | | QPS16358 - Inverted) | crystalline precipitate | crystalline precipitate |
| | | AM003 v10 | 3 Months | Clear, colourless | Clear, colourless |
| | | | (QPS16554 - Upright, | solution free from | solution free from |
| | | | QPS16558 - Inverted) | crystalline precipitate⁹ | crystalline precipitate |
| | | AM003 v10 | 6 Months | Clear, colourless | Clear, colourless |
| | | | (QPS17288 - Upright, | solution free from | solution free from |
| | | | QPS17289 - Inverted) | crystalline precipitate | crystalline precipitate |
| | | AM003 v10 | 9 Months | Clear, colourless | Clear, colourless |
| | | | (QPS17938 - upright, | solution free from | solution free from |
| | | | QPS17939 - inverted) | crystalline precipitate¹⁰ | crystalline precipitate¹⁰ |
| | | AM003 v10 | 12 Months | Clear, colourless | Clear, colourless |
| | | | (QPS18541 - inverted, | solution free from | solution free from |
| | | | QPS18542 - upright) | crystalline precipitate | crystalline precipitate |
| | | AM003 v11 | 18 Months | Clear, colourless | Clear, colourless |
| | | | (QPS20005 - Upright, | solution free from | solution free from |
| | | | QPS20006 - Inverted) | crystalline precipitate | crystalline precipitate |
| | | AM003v11 | 24 Months | Clear, colourless | Clear, colourless |
| | | | (QPS21433 - upright, | solution free from | solution free from |
| | | | QPS21434 - Inverted) | crystalline precipitate | crystalline precipitate |

Table 25 illustrates that following storage at 25° C. and 60% relative humidity, after 1 month and 2 months the liquid pharmaceutical composition comprising gemfibrozil samples were clear, colorless, and free from crystalline precipitate; after 3 months the samples were clear, pale yellow, and free from crystalline precipitate; after 6 months and 9 months the samples were clear, yellow, and free from crystalline precipitate; and after 12 months, 18 months, and 24 months the samples were clear, pale yellow, and free from crystalline precipitate.

TABLE 25

| Storage | | | Time Point | | |
|---|---|---|---|---|---|
| Condition | Specification | Method | (Months) | Result | |
| 25° C./60 | Clear, colourless to | AM003 v10 | 1 Month | Clear, colourless | Clear, colourless |
| % RH | pale yellow, | | (QPS16083-Upright, | solution free from | solution free from |
| | solution, free from | | QPS16084-Inverted) | crystalline precipitate⁸ | crystalline precipitate⁸ |
| | crystalline | AM003 v10 | 2 Months | Clear, colourless | Clear, colourless |
| | precipitate.⁵ | | (QPS16291-Upright, | solution free from | solution free from |
| | | | QPS16292-Inverted) | crystalline precipitate | crystalline precipitate |
| | | AM003 v10 | 3 Months | Clear, colourless | Clear, colourless |
| | | | (QPS16566 - Upright, | solution free from | solution free from |
| | | | QPS16569 - Inverted) | crystalline precipitate | crystalline precipitate |
| | | AM003 v10 | 6 Months | Clear, colourless | Clear, colourless |
| | | | (QPS17290 - Upright, | solution free from | solution free from |
| | | | QPS17291 - Inverted) | crystalline precipitate | crystalline precipitate |
| | | AM003 v10 | 9 Months | Clear, colourless | Clear, colourless |
| | | | (QPS17940-upright) | solution free from | solution free from |
| | | | (QPS17941-Inverted) | crystalline precipitate¹⁰ | crystalline precipitate¹⁰ |
| | | | | Inverted | Upright |
| 25° C./60 | Clear, colourless to | AM003 v10 | 12 Months | Clear, colourless | Clear, colourless |
| % RH | pale yellow, | | (QPS18543) - inverted | solution free from | solution free from |
| | solution, free from | | QPS18544 - upright) | crystalline precipitate | crystalline precipitate |
| | crystalline | AM003 v10 | 18 Months | Clear, colourless | Clear, colourless |
| | precipitate.⁵ | | (QPS20007 - Upright, | solution free from | solution free from |
| | | | QPS20008 - Inverted) | crystalline precipitate | crystalline precipitate |
| | | AM003 v10 | 24 Months | Clear, colourless | Clear, colourless |
| | | | (QPS21435 - Upright, | solution free from | solution free from |
| | | | QPS21436 - Inverted) | crystalline precipitate | crystalline precipitate |

Table 26 illustrates that following storage at 40° C. and 75% relative humidity, after 1 month the samples were clear, yellow, and free from crystalline precipitate; after 2 months and 3 months the samples were clear, brown, and free from crystalline precipitate; and after 6 months the liquid pharmaceutical composition comprising gemfibrozil samples were clear, yellow, and free from crystalline precipitate.

TABLE 26

| Storage | | | Time Point | | |
|---|---|---|---|---|---|
| Condition | Specification | Method | (Months) | Result | |
| 40° C./75 | Clear, colourless | AM003 v10 | 1 Month | Clear, colourless | Clear, colourless |
| % RH | to pale yellow, | | (QPS16085- Upright, | solution free from | solution free from |
| | solution, free | | QPS16086- Inverted) | crystalline precipitate⁸ | crystalline precipitate⁸ |
| | from crystalline | AM003 v10 | 2 Months | Clear, colourless | Clear, colourless |
| | precipitate.⁵ | | (QPS16293- Upright, | solution free from | solution free from |
| | | | QPS16294- Inverted) | crystalline precipitate¹¹ | crystalline precipitate¹¹ |
| | | AM003 v10 | 3 Months | Clear, colourless | Clear, colourless |
| | | | (QPS16566 - Upright, | solution free from | solution free from |
| | | | QPS16569 - Inverted) | crystalline precipitate¹¹ | crystalline precipitate¹¹ |
| | | AM003 v10 | 6 Months | Clear, colourless | Clear, colourless |
| | | | (QPS17292 - Upright, | solution free from | solution free from |
| | | | QPS17293 - Inverted) | crystalline precipitate | crystalline precipitate |

Table 27 below illustrates the stability of gemfibrozil in the liquid pharmaceutical composition following storage at 2-8° C. in either an inverted or upright position. The assay demonstrated that all samples were stable during storage in either position until 24 months. For example, at 18 months 96.9% of the gemfibrozil was stable following incubation of the liquid pharmaceutical composition in the inverted position at 2-8° C. and at 24 months 99.6% of the gemfibrozil was stable following incubation of the liquid pharmaceutical composition in the inverted position at 2-8° C. Similar results were observed at all storage time points in either storage position as illustrated in Table 27. All samples complied with the specification were within the alarm limits.

TABLE 27

| Storage | | | Time Point | | |
|---|---|---|---|---|---|
| Condition | Specification | Method | (Months) | Result | |
| Initial | 90.0-110.0% | AM324 v10 | Intial¹² | R1 = 99.5% | |
| | | (Draft) | (QPS15848) | R2 = 99.4% | |
| | | | | Mean = 99.4% | |
| | | | | Inverted | Upright |
| 2-8° C. | 90.0-110.0% | AM324 v11 | 1 Month | R1 = 107.0% | R1 = [symbol] % |
| | | (Draft) | (QPS16081- Upright, | R2 = 106.9% | R2 = [symbol] % |
| | | | QPS16082- Inverted) | Mean = 107.0% [symbol] | Mean = [symbol] % |
| | | AM324 v11 | 2 Months | R1 = 99.4% | R1 = [symbol] % |
| | | (Draft) | (QPS16289- Upright, | R2 = [symbol] % | R2 = 99.5% |
| | | | QPS16290- Inverted) | Mean = [symbol] % | Mean = 99.5% |
| | | AM324 v01 | 3 Months¹⁴ | R1 = 100.6% | R1 = 98.1% |
| | | | (QPS16564- Upright, | R2 = 100.9% | R2 = 98.4% |
| | | | QPS16558- Inverted) | Mean = 100.7% | Mean = 98.3% |
| | | AM324 v01 | 6 Months [symbol] | R1 = [symbol] % | R1 = [symbol] % |
| | | | (QPS172 [symbol] -Upright, | R2 = [symbol] % | R2 = [symbol] % |
| | | | QPS17289 - Inverted) | Mean = [symbol] % | Mean = [symbol] % |
| | | AM324 v02 | 9 Months | R1 = 99.5% | R1 = 99.7% |
| | | | (QPS17938-Upright) | R2 = 99.3% | R2 = 99.8% |
| | | | (QPS17939-Inverted) | Mean = 99.4% | Mean = [symbol] % |
| | | AM324 v02 | 12 Months | R1 = 99.0% | R1 = [symbol] % |
| | | | (QPS18541 - Inverted | R2 = [symbol] % | R2 = [symbol] % |
| | | | QPS18542 - Upright) | Mean = 99.1% | Mean = [symbol] % |
| | | AM324 v02 | 18 Months | R1 = 96.9% | R1 = [symbol] % |
| | | | (QPS20005 - Upright, | R2 = 96.9% | R2 = [symbol] % |
| | | | QPS20006 - Inverted) | Mean = [symbol] % | Mean = [symbol] % |
| | | AM324 v02 | 24 Months | R1 = [symbol] % | R1 = 98.9% |
| | | | (QPS21433 - upright | R2 = 99.5% | R2 = 99.2% |
| | | | QPS21434 - Inverted) | Mean = 99.6% | Mean = 99.0% |

[symbol]

indicates data missing or illegible when filed

Table 28 below illustrates the stability of gemfibrozil in the liquid pharmaceutical composition following storage at 25° C. in either an inverted or upright position. The assay demonstrated that all samples were stable during storage in either position until 24 months. For example, at 18 months 96.9% of the gemfibrozil was stable following incubation of the liquid pharmaceutical composition in the inverted position at 25° C. and at 24 months 99.5% of the gemfibrozil was stable following incubation of the liquid pharmaceutical composition in the inverted position at 25° C. Similar results were observed at all storage time points in either storage position as illustrated in Table 28. All samples complied with the specification were within the alarm limits.

TABLE 28

| Storage | | | Time Point | Result | Results |
|---|---|---|---|---|---|
| Condition | Specification | Method | (Months) | Inverted | Upright |
| 25° C./60% RH | 90.0-110.0% | AM324 v11 | 1 Month | R1 = [symbol] % | R1 = [symbol] % |
| | | (Draft) | (QPS [symbol] - Upright, | R2 = [symbol] % | R2 = [symbol] % |
| | | | QPS160 [symbol] - Inverted) | Mean = [symbol] % | Mean = [symbol] % |
| | | AM324 v11 | 2 Months | R1 = 99.5% | R1 = [symbol] % |
| | | (Draft) | (QPS16291- Upright, | R2 = [symbol] % | R2 = [symbol] % |
| | | | QPS [symbol] - Inverted) | Mean = [symbol] % | Mean = [symbol] % |
| | | AM324 v01 | 3 Months [symbol] | R1 = [symbol] % | R1 = [symbol] % |
| | | | (QPS [symbol] - Upright, | R2 = [symbol] % | R2 = [symbol] % |
| | | | QPS [symbol] - Inverted) | Mean = [symbol] % | Mean = [symbol] % |
| | | AM324 v01 | 6 Months [symbol] | R1 = [symbol] % | R1 = [symbol] % |
| | | | (QPS17290 -Upright, | R2 = [symbol] % | R2 = [symbol] % |
| | | | QPS17 [symbol] - Inverted) | Mean = [symbol] % | Mean = [symbol] % |
| 25° C./80% RH | 90.0-110.0% | AM324 v02 | [symbol] Months | R1 = 100.1% | R1 = [symbol] % |
| | | | (QPS [symbol] -Upright) | R2 = 100.0% | R2 = 99.3% |
| | | | (QPS [symbol] -Inverted) | Mean = 100.0% | Mean = [symbol] % |
| | | AM324 v02 | 12 Months | R1 = [symbol] % | R1 = [symbol] % |
| | | | (QPS [symbol] - Inverted | R2 = [symbol] % | R2 = [symbol] % |
| | | | QPS [symbol] - Upright) | Mean = [symbol] % | Mean = [symbol] % |
| | | AM324 v02 | 18 Months | R1 = [symbol] % | R1 = [symbol] % |
| | | | (QPS20007 - Upright, | R2 = v% | R2 = [symbol] % |
| | | | QPS20008 - Inverted) | Mean = [symbol] % | Mean = [symbol] % |
| | | AM324 v02 | 24 Months [symbol] | R1 = [symbol] % | R1 = [symbol] % |
| | | | (QPS21435 - upright | R2 = [symbol] % | R2 = [symbol] % |
| | | | QPS21436 - Inverted) | Mean = [symbol] % | Mean = [symbol] % |

[symbol]

indicates data missing or illegible when filed

Table 29 below illustrates the stability of gemfibrozil in the liquid pharmaceutical composition following storage at 40° C. in either an inverted or upright position. The assay demonstrated that all samples were stable during storage in either position until 6 months. For example, at 3 months 98.8% of the gemfibrozil was stable following incubation of the liquid pharmaceutical composition in the inverted position at 40° C. and at 24 months 99.3% of the gemfibrozil was stable following incubation of the liquid pharmaceutical composition in the inverted position at 40° C. Similar results were observed at all storage time points in either storage position as illustrated in Table 29. All samples complied with the specification and were within the alarm limits.

TABLE 29

| Storage | | | Time Point | Result | Results |
|---|---|---|---|---|---|
| Condition | Specification | Method | (Months) | Inverted | Upright |
| 40° C./75% RH | 90.0-110.0% | AM324 v11 | 1 Month | R1 = [symbol] % | R1 = [symbol] % |
| | | (Draft) | (QPS [symbol] - Upright, | R2 = 99.1% | R2 = 99.0% |
| | | | QPS [symbol] - Inverted) | Mean = 99.1% | Mean = 99.0% |
| | | AM324 v11 | 2 Months | R1 = [symbol] % | R1 = [symbol] % |
| | | (Draft) | (QPS16293- Upright, | R2 = 99.7% | R2 = [symbol] % |
| | | | QPS16294 - Inverted) | Mean = [symbol] % | Mean = [symbol] % |
| | | AM324 v01 | 3 Months¹⁴ | R1 = [symbol] % | R1 = [symbol] % |
| | | | (QPS [symbol] - Upright, | R2 = [symbol] % | R2 = 99.1 |
| | | | QPS [symbol] - Inverted) | Mean = [symbol] % | Mean = [symbol] % |
| | | AM324 v01 | 6 Months [symbol] | R1 = 99.2% | R1 = 99.1% |
| | | | (QPS17292 -Upright, | R2 = [symbol] % | R2 = 99.2% |
| | | | QPS17293 - Inverted) | Mean = [symbol] % | Mean = 99.1% |

[symbol]

indicates data missing or illegible when filed

HPLC was used determine if the gemfibrozil peak(s) decreased and/or whether new substances were present in the liquid pharmaceutical composition. No related substances were detected within specification limits following storage of the liquid pharmaceutical composition comprising gemfibrozil at 2-8° C. or 25° C. from the initial time point until 24 months in either the inverted or upright position. No related substances were detected within specification limits following storage of the liquid pharmaceutical composition comprising gemfibrozil at 40° C. from the initial time point until 6 months in either the inverted or upright position. Specification limits allowed for the formation of gemfibrozil-related substances from the initial timepoint throughout the duration of the time periods described herein; however, total impurities should have an increase of <1.00%, and any individual impurities should have an increase<0.20% of the correspondence peak area.

Density of all samples of the liquid pharmaceutical composition comprising gemfibrozil ranged between 1.010 g/cm³-1.012 g/cm³. The density values were consistent regardless of storage temperature (2-8° C., 25° C., or 40° C.), duration (1 month, 2 months, 3 months, 6 months, months, 12 months, 18 months, or 24 months), or position (inverted or upright). Data was within protocol alarms for all storage conditions.

Table 30 below illustrates the pH of the liquid pharmaceutical composition comprising gemfibrozil following storage at 2-8° C. in either an inverted or upright position. Under all storage times and conditions, the pH values ranged from pH 10.47-pH 10.67. For example, the pH of the liquid pharmaceutical composition comprising gemfibrozil was 10.52 at 18 months and 10.58 at 24 months following storage in the inverted position at 2-8° C. Similar results were observed for all storage time points in either storage position as illustrated in Table 30. There was no trend in pH values throughout the study and all pH values were within the alarm limits of the study (+/−0.5 units).

TABLE 30

| Storage | | | Time Point | | |
|---|---|---|---|---|---|
| Condition | Specification | Method | (Months) | Result | |
| Initial | Read and | AM020 v06 | Initial | 10.39 | |
| | Record | | (QPS15848) | | |
| | | | | Inverted | Upright |
| 2-3° C. | Read and | AM020 v06 | 1 Month | 10.56 | 10.56 |
| | Record | | (QPS16081 -Upright, | | |
| | | | QPS16082 - Inverted) | | |
| | | AM020 v07 | 2 Months | 10.67 ^{17, 18} | [symbol] ^{17, 18} |
| | | | (QPS16289 - Upright | | |
| | | | QPS [symbol] - Inverted) | | |
| | | AM020 v07 | [symbol] Months | 10.63 ^{17, 18} | 10.64 ^{17, 18} |
| | | | (QPS [symbol] - Upright | | |
| | | | QPS [symbol] - Inverted) | | |
| | | AM020 v07 | 6 Months | 10.57 | 10.57 |
| | | | (QPS [symbol] - Upright | | |
| | | | QPS [symbol] - Inverted) | | |
| | | AM020 v08 | 9 Months | 10.62 | 10.59 |
| | | | (QPS [symbol] - upright | | |
| | | | QPS [symbol] - Inverted) | | |
| | | AM020 v08 | 12 Months | 10.47 | 10.47 |
| | | | (QPS18541 - Inverted | | |
| | | | QPS18542 - Upright) | | |
| | | AM020 v08 | 18 Months | 10.52 | 10.52 |
| | | | (QPS20005 - Upright | | |
| | | | QPS20006 - Inverted) | | |
| | | AM020 v08 | 24 Months | 10.58 | 10.57 |
| | | | (QPS21433 - Upright | | |
| | | | Q2521434 -Invested) | | |

[symbol]

indicates data missing or illegible when filed

Table 31 below illustrates the pH of the liquid pharmaceutical composition comprising gemfibrozil following storage at 25° C. in either an inverted or upright position. Under all storage times and conditions, the pH values ranged from pH 10.34-pH 10.56. For example, the pH of the liquid pharmaceutical composition comprising gemfibrozil was 10.38 at 18 months and 10.44 at 24 months following storage in the inverted position at 25° C. Similar results were observed for all storage time points in either storage position as illustrated in Table 31. There was no trend in pH values throughout the study and all pH values were within the alarm limits of the study (+/−0.5 units).

TABLE 31

| Storage | | | Time Point | Result | Results |
|---|---|---|---|---|---|
| Condition | Specification | Method | (Months) | Inverted | Upright |
| 25° C./60% RH | Read and | AM020 v06 | 1 Month | 10.51 | 10.52 |
| | Record | | (QPS16083 -Upright, | | |
| | | | QPS16084 - Inverted) | | |
| | | AM020 v07 | 2 Months | 10.45 [symbol] | 10.35 [symbol] |
| | | | (QPS [symbol] - Upright | | |
| | | | QPS [symbol] - Inverted) | | |
| | | AM020 v07 | 1 Months | 10.53 [symbol] | [symbol] |
| | | | (QPS [symbol] - Upright | | |
| | | | QPS [symbol] - Inverted) | | |
| | | AM020 v07 | 6 Months | 10.48 | 10.48 |
| | | AM020 v0 [symbol] | 9 Months | 10.48 | 10.48 |
| | | | (QPS17940 - upright | | |
| | | | QPS17941 - Inverted) | | |
| | | AM020 v0 [symbol] | 12 Months | 10.34 | 10.34 |
| | | | (QPS [symbol] - Inverted | | |
| | | | QPS [symbol] - Upright) | | |
| | | AM020 v0 [symbol] | 18 Months | [symbol] | [symbol] |
| | | | (QPS20007 - Upright | | |
| | | | QPS [symbol] - Inverted) | | |
| | | AM020 v0 [symbol] | 24 Months | 10.44 | 10.44 |
| | | | (QPS21435 - Upright | | |
| | | | Q2521435 -Invested) | | |

[symbol]

indicates data missing or illegible when filed

Table 32 below illustrates the pH of the liquid pharmaceutical composition comprising gemfibrozil following storage at 40° C. in either an inverted or upright position. Under all storage times and conditions, the pH values ranged from pH 10.35-pH 10.49. For example, the pH of the liquid pharmaceutical composition comprising gemfibrozil was 10.47 at 1 month and 10.40 at 6 months following storage in the inverted position at 40° C. Similar results were observed for all storage time points in either storage position as illustrated in Table 32. There was no trend in pH values throughout the study and all pH values were within the alarm limits of the study (+/−0.5 units).

TABLE 32

| Storage | | | Time Point | Result | Results |
|---|---|---|---|---|---|
| Condition | Specificati | Method | (Months) | Inverted | Upright |
| 40° C./75% RH | Read and | AM020 v [symbol] | 1 Month | 10.41 | 10.42 |
| | Record | | (QPS [symbol] -Upright, | | |
| | | | QPS [symbol] - Inverted) | | |
| | | AM020 v [symbol] | 2 Months | [symbol] ¹⁸ | [symbol] ¹⁸ |
| | | | (QPS [symbol] - Upright | | |
| | | | QPS [symbol] - Inverted) | | |
| | | AM020 v [symbol] | 1 Months | 10.47 ¹⁸ | [symbol] ¹⁸ |
| | | | (QPS [symbol] - Upright | | |
| | | | QPS [symbol] - Inverted) | | |
| | | AM020 v [symbol] | 6 Months | 10.40 | 10.41 |
| | | | (QPS17292 - Upright | | |
| | | | QPS [symbol] - Inverted) | | |

[symbol]

indicates data missing or illegible when filed

Microbial purity was determined by assessing the potential growth of Escherichia Coli (E. coli), total aerobic microbial count (TAMC), and total yeast/mold count (TYMC) according to current Ph Eur 9.8 (2.6.12, 2.6.13) and USP 42<61><62> test method for total count and detection of specified organisms in the liquid pharmaceutical composition comprising gemfibrozil.

Table 33 below illustrates total aerobic microbial count, total E. coli count, and the total combined yeast and mold count of the liquid pharmaceutical composition comprising gemfibrozil following storage at 2-8° C. and 25° C. at 60% relative humidity, in either an inverted or upright position. Following 12 months or 24 months of storage at either storage temperature, in either position, total aerobic microbial count, total E. coli count, and the total combined yeast and mold count of the liquid pharmaceutical composition comprising gemfibrozil demonstrated acceptably low levels of microbes and remained within the alarm limits of the study (absence of E. Coli in 1 ml, less than 10 cfu/ml TAMC, and less than 10 cfu/ml TYMC according to current USP 42 <61><62> test method for total count and detection of specified organisms).

TABLE 33

| Storage | | | Time Point | | |
|---|---|---|---|---|---|
| Condition | Specification | Method | (Months) | Result | |
| Initial | Complies with | USP <61> | Initial | TAMC [symbol] <10 cfu/mL | |
| | Ph. Eur 5.1.4 | and <62> | (QPS15845) | TYMC [symbol] <10 cfu/mL | |
| | and USP | RSSL method | | E. coli Absent in 1 mL | |
| | Chapter [symbol] | reference: | | | |
| | TAMC [symbol] | [symbol] | | | |
| | CFU/mL | | | | |
| | TYMC [symbol] | | | | |
| | CFU/mL | | | | |
| | Absence of E. coli | | | | |
| | in 1 mL | | | | |
| | | | | Inverted | Upright |
| | | USP <61> | 12 Months | TAMC [symbol] <10 cfu/mL | TAMC [symbol] <10 cfu/mL |
| | | and <62> | (QPS18541 - Inverted | TYMC [symbol] <10 cfu/mL | TYMC [symbol] <10 cfu/mL |
| | | RSSL method | QPS18542 - Upright | E. coli Absent | E. coli Absent |
| | | reference: | | in 1 mL | in 1 mL |
| | | [symbol] | | | |
| | | USP <61> | 24 Months | TAMC [symbol] <10 cfu/mL | TAMC [symbol] <10 cfu/mL |
| | | and <62> | (QPS21433 - Inverted | TYMC [symbol] <10 cfu/mL | TYMC [symbol] <10 cfu/mL |
| | | RSSL method | QPS21434 - Upright | E. coli Absent | E. coli Absent |
| | | reference: | | in 1 mL | in 1 mL |
| | | [symbol] | | | |
| | | USP <61> | 12 Months | TAMC [symbol] <10 cfu/mL | TAMC [symbol] <10 cfu/mL |
| | | and <62> | (QPS18543 - Inverted | TYMC [symbol] <10 cfu/mL | TYMC [symbol] <10 cfu/mL |
| | | RSSL method | QPS18544 - Upright | E. coli Absent | E. coli Absent |
| | | reference: | | in 1 mL | in 1 mL |
| | | [symbol] | | | |
| | | USP <61> | 24 Months | TAMC [symbol] <10 cfu/mL | TAMC [symbol] <10 cfu/mL |
| | | and <62> | (QPS [symbol] - Inverted | TYMC [symbol] <10 cfu/mL | TYMC [symbol] <10 cfu/mL |
| | | RSSL method | QPS [symbol] - Upright | E. coli Absent | E. coli Absent |
| | | reference: | | in 1 mL | in 1 mL |
| | | [symbol] | | | |

[symbol]

indicates data missing or illegible when filed

Preservative efficacy was determined by assessing the potential growth rates of bacteria and yeast/mold over time according to current USP 42 <51> test method in the liquid pharmaceutical composition comprising gemfibrozil.

Table 34 below illustrates a bacterial growth rate≥1 log reduction from 0 days to 14 days, with no increase from 14 days to 28 days, and no increase in yeast and molds from 0 days to 14 days and from 0 days to 28 days in the liquid pharmaceutical composition comprising gemfibrozil following storage at 2-8° C. and 25° C. at 60% relative humidity, in either an inverted or upright position. Following 12 months or 24 months of storage at either storage temperature, in either position, total aerobic microbial count, total E. coli count, and the total combined yeast and mold count of the liquid pharmaceutical composition comprising gemfibrozil complied with the current USP 42<51> test method and remained within the alarm limits of the study (bacterial growth rate≥1 log reduction from 0 days to 14 days, with no increase from 14 days to 28 days, and no increase in yeast and molds from 0 days to 14 days and from 0 days to 28 days).

TABLE 34

| Storage | | | Time Point | | |
|---|---|---|---|---|---|
| Condition | Specification | Method | (Months) | Result | |
| Initial | Complies with | USP < [symbol] > | Initial | Complies with USP <51> | |
| | USP < [symbol] > | RSSL method | (QPS [symbol] ) | | |
| | Bacteria [symbol] log | reference: | | | |
| | reduction [symbol] | [symbol] | | | |
| | increase from | | | | |
| | T = 14 to 28 d | | | | |
| | [symbol] | | | | |
| | no increase from | | | | |
| | [symbol] | | | | |
| | | | | Inverted | Upright |
| | | USP <51> | 12 Months | Complies with | Complies with |
| | | RSSl method | (QPS [symbol] - Inverted | USP <51> | USP <51> |
| | | reference: | QPS [symbol] - Upright | | |
| | | P19- [symbol] | | | |
| | | USP <51> | 16 Months | Complies with | Complies with |
| | | RSSl method | (QPS20006 - Inverted | USP <51> | USP <51> |
| | | reference: | QPS [symbol] - Upright | | |
| | | P19- [symbol] | | | |
| | | USP <51> | 24 Months | Complies with | Complies with |
| | | RSSl method | (QPS21433 - Inverted | USP <51> | USP <51> |
| | | reference: | QPS21434 - Upright | | |
| | | P19- [symbol] | | | |
| [symbol] RH | | USP <51> | 12 Months | Complies with | Complies with |
| | | RSSl method | (QPS18701 - Inverted | USP <51> | USP <51> |
| | | reference: | QPS [symbol] - Upright | | |
| | | P19- [symbol] | | | |
| | | USP <51> | 16 Months [symbol] | Complies with | Complies with |
| | | RSSl method | (QPS20007 - Inverted | USP <51> | USP <51> |
| | | reference: | QPS20008 - Upright | | |
| | | P19- [symbol] | | | |
| | | USP <51> | 24 Months | To be reported in | To be reported in |
| | | RSSl method | (QPS [symbol] - Inverted | version [symbol] | version [symbol] |
| | | reference: | QPS [symbol] - Upright | | |
| | | P19- [symbol] | | | |

[symbol]

indicates data missing or illegible when filed

Preservative efficacy was also determined by assessing for the presence of Burkholderia capacia complex (BCC) over time according to current USP <60> test method in the liquid pharmaceutical composition comprising gemfibrozil.

Table 35 below illustrates that the liquid pharmaceutical composition comprising gemfibrozil following storage at 2-8° C. and 25° C. at 60% relative humidity, in either an inverted or upright position complies with the current USP <60> test method. Following 12 months or 24 months of storage at either storage temperature, in either position, growth in the liquid pharmaceutical composition comprising gemfibrozil complied with the current USP <60> test method and remained within the alarm limits of the study.

TABLE 35

| Storage | | | Time Point | Result | |
|---|---|---|---|---|---|
| Condition | Specification | Method | (Months) | Inverted | Upright |
| 2-8° C. | Complies with | USP < [symbol] > | 12 Months | Complies with | Complies with |
| | USP < [symbol] > | RSSl method | (QPS18541 - Inverted | USP < [symbol] > | USP <60> |
| | | reference: | QPS18542 - Upright | | |
| | | P19- [symbol] | | | |
| | | USP < [symbol] > | 24 Months | Complies with | Complies with |
| | | RSSl method | (QPS21433 - Inverted | USP < [symbol] > | USP <60> |
| | | reference: | QPS21434 - Upright | | |
| | | P19- [symbol] | | | |
| 25° C./ [symbol] % RH | | USP <60> | 12 Months | Complies with | Complies with |
| | | RSSl method | (QPS18543 - Inverted | USP < [symbol] > | USP < [symbol] > |
| | | reference: | QPS18544 - Upright | | |
| | | P19- [symbol] | | | |
| | | USP <60> | 24 Months | Complies with | Complies with |
| | | RSSl method | (QPS21435 - Inverted | USP < [symbol] > | USP <60> |
| | | reference: | QPS21436 - Upright | | |
| | | P19- [symbol] | | | |

[symbol]

indicates data missing or illegible when filed

Liquid pharmaceutical composition placebo to match gemfibrozil control samples were also prepared in a carbonate buffer having a pH of 10.5 (+/−0.5) to support the gemfibrozil studies. Samples were evaluated at 1 month, 2 months, 3 months, 6 months, 9 months, 12 months, 18 months, and 24 months after preparation under storage conditions of approximately 2-8° C. or 25° C. with 60% relative humidity. Samples were also evaluated at 1 month, 2 months, 3 months, and 6 months after preparation at 40° C. with 75% relative humidity. All formulation samples were stored either upright or inverted for the indicated time period. No preservatives or other cosolvents/surfactants were included in these placebo formulations.

Following preparation of the liquid pharmaceutical composition placebo to match gemfibrozil samples, the samples were colorless to yellow, free from precipitate, and had the aroma of cherry. Following storage at 2-8° C., after all time points the samples were clear, colorless, and were free from crystalline precipitate. Following storage at 25° C. and 60% relative humidity, after 1 month and 2 months the liquid pharmaceutical composition placebo to match gemfibrozil samples were clear, colorless, and free from crystalline precipitate; after 3 months to 18 months the liquid pharmaceutical composition placebo to match gemfibrozil samples were clear, pale yellow, and free from crystalline precipitate, with the exception of the 6-month time point in the upright sample which was clear, yellow, and free from crystalline precipitate. Following storage at 40° C. and 75% relative humidity, after 1 the liquid pharmaceutical composition placebo to match gemfibrozil samples were clear, yellow, and free from crystalline precipitate; after 2 months and 3 months the liquid pharmaceutical composition placebo to match gemfibrozil samples were clear, brown, and free from crystalline precipitate; and after 6 months the liquid pharmaceutical composition placebo to match gemfibrozil samples were clear, yellow, and free from crystalline precipitate.

### Example 11

The stability of the liquid pharmaceutical composition comprising gemfibrozil (15 mg/ml) in a carbonate buffer having a pH of 10.5 (+/−0.5) was evaluated under temperature strain conditions according to Table 36 below. No preservatives or other cosolvents/surfactants were included in these F_{CARB15} formulations.

TABLE 36

| | Working Day Number | | | | | | | |
|---|---|---|---|---|---|---|---|---|
| | 1 | 2 | 3 | 4 | 5 | 6 | 7 | 8 |
| Notes on | N/A | N/A | Can be | N/A | Can be | These three days must be | | |
| timing | | | Frozen | | held | consecutive (i.e. no weekend in | | |
| | | | over a | | over a | between Working Day 6 and 8) | | |
| | | | weekend | | weekend | | | |
| Hold | −20° C. | 25° C. | −20° C. | 25° C. | −20° C. | 25° C. | | |
| overnight | | | | | | | | |
| at: | | | | | | | | |
| Sampling | Inspection | None | Inspection | None | Inspection | None | None | Inspection |
| and | only: Step | | only: Step | | only: Step | | | and assay |
| Inspection | 3 | | 3 | | 3 | | | sampling: |
| | | | | | | | | Step 8 |

The appearance of the active samples (A1 and A2; having gemfibrozil) and the placebo samples (P1 and P2) was assessed prior to and post freezing at −20° C. Before the first freeze, all sample solutions were clear, transparent liquid without visible crystals. No haziness or particulates/fibers was observed. Before the second freeze (Day 3) and throughout the rest of the freeze-thaw cycles all samples had some fibers that appeared non-crystalline. No color change was observed for the samples at any of the time points.

The pH values for the samples that underwent the freeze-thaw cycling shown in Table 36 were compared with control placebo and control gemfibrozil samples that did not undergo temperature cycling. As shown in Table 37, the pH of P1 and P2 was 0.07-0.06 pH units less than the placebo control sample, and the pH of A1 and A2 was 0.05-0.06 pH units less that the gemfibrozil control sample. Although the pH of the gemfibrozil samples was approximately 0.2 pH units less than the pH of the placebo samples, temperature cycling did not appear to have an effect on the pH stability of the samples.

TABLE 37

| Sample | pH |
|---|---|
| Placebo Control | 10.48 |
| P1 | 10.42 |
| P2 | 10.41 |
| Active Control | 10.24 |
| A1 | 10.19 |
| A2 | 10.18 |

The stability of gemfibrozil was assessed in each of the samples following the freeze-thaw cycling shown in Table 38. Gemfibrozil assay results of all samples complied with the specification. Assay results of all samples were within the alarm limits (within 90.0%-110.0%).

TABLE 38
ASSAY RESULTS

| | | | | | Replicate |
|---|---|---|---|---|---|
| Bottle Number | | Rep 1 | Rep 2 | Mean | Agreement |
| Active | Assay | 14.93 | 14.92 | 14.92 | 0.1 |
| Control | (mg/mL) | | | | |
| | % Recovery | 99.6 | 99.4 | 99.5 | |
| A1 | Assay | 15.07 | 15.06 | 15.07 | 0.1 |
| | (mg/mL) | | | | |
| | % Recovery | 100.5 | 100.4 | 100.4 | |
| A2 | Assay | 14.91 | 14.93 | 14.92 | 0.1 |
| | (mg/mL) | | | | |
| | % Recovery | 99.4 | 99.5 | 99.5 | |

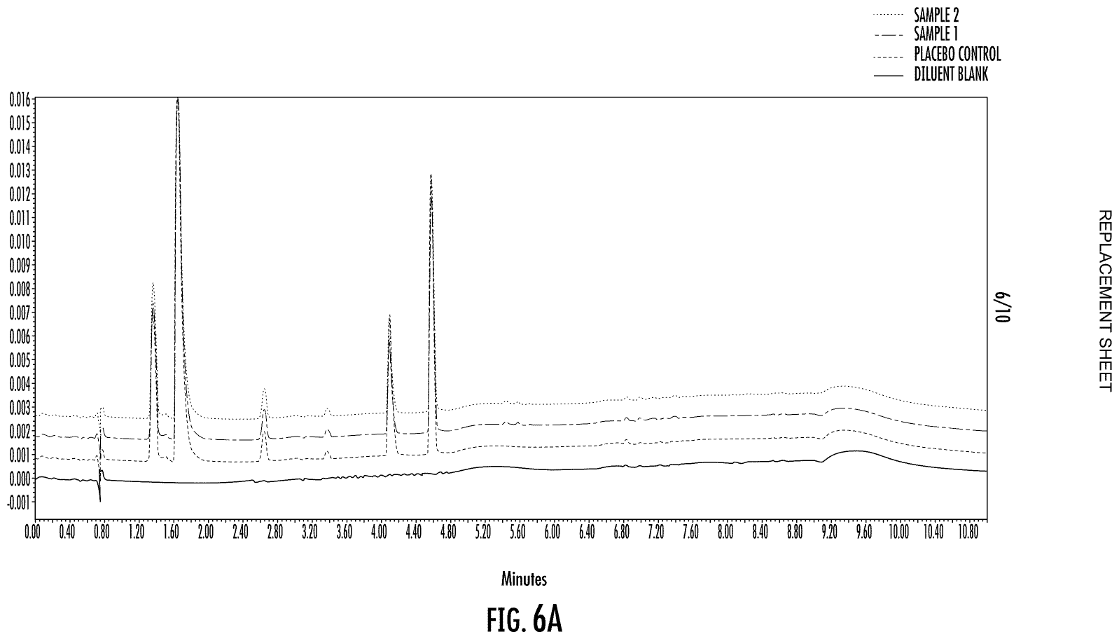

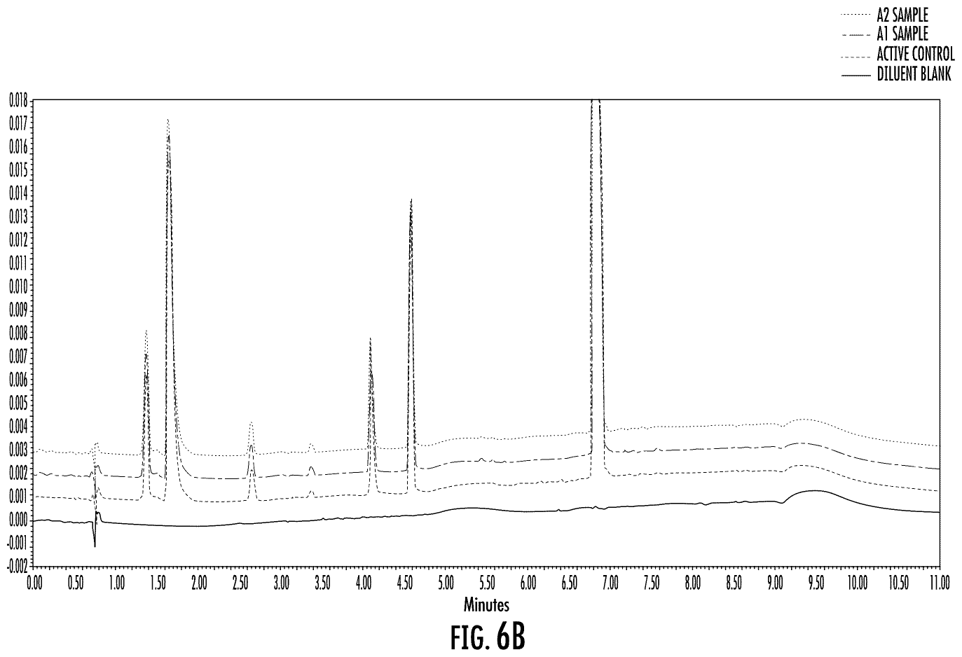

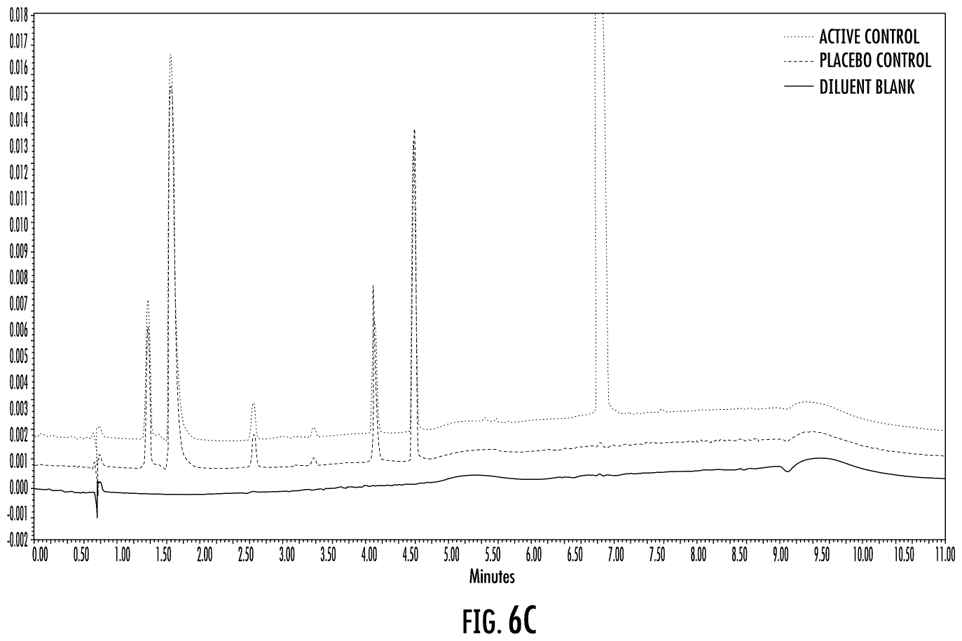

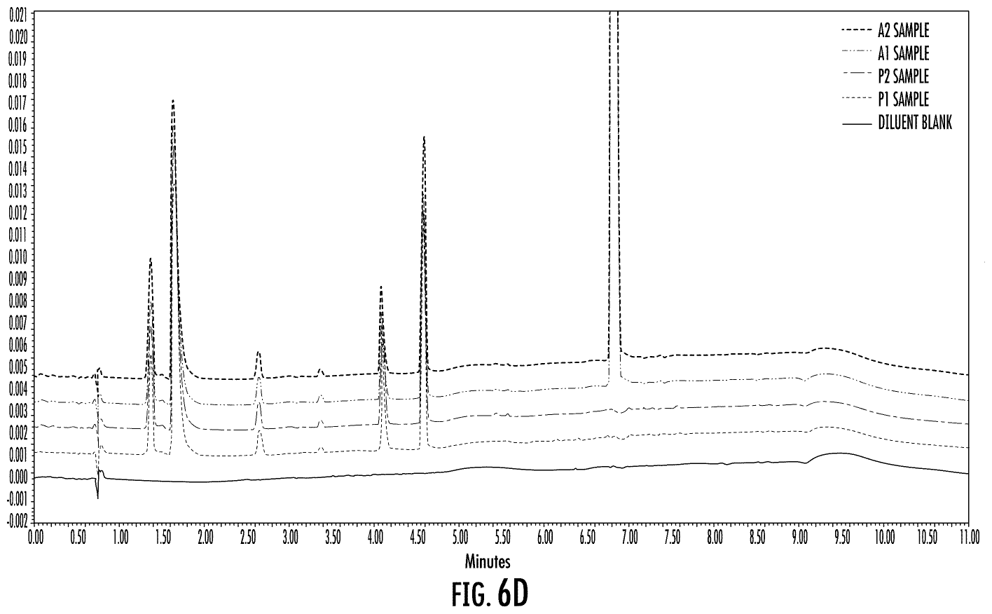

As shown in FIGS. 6A-6D, there were no detectable related substances in the control samples that had not undergone freeze-thaw cycles or the samples that had undergone the freeze-thaw cycle (both placebo and gemfibrozil). In FIG. 6A, the bottom line represents the diluent blank sample, the second line from the bottom represents the placebo control sample, the third line from the bottom represents the P1 sample, and the top line represents the P2 sample. The overlay graph demonstrates that there are no impurities above LOQ between any of the placebo samples. In FIG. 6B, the bottom line represents the diluent blank sample, the second line from the bottom represents the active control sample, the third line from the bottom represents the A1 sample, and the top line represents the A2 sample. The overlay graph demonstrates that there are no impurities above LOQ between any of the active (gemfibrozil) samples. In FIG. 6C, the bottom line represents the diluent blank sample, the second line from the bottom represents the placebo control sample, and the top line represents the active control sample. The overlay graph demonstrates that there are no impurities above LOQ between any of the active control samples, placebo or gemfibrozil. In FIG. 6D, the bottom line represents the diluent blank sample, the second line from the bottom represents the P1 sample, the third line from the bottom represents the P2 sample, the fourth line from the bottom represents the A1 sample, and the top line represents the A2 sample. The overlay graph demonstrates that there are no impurities above LOQ between any of the active (gemfibrozil) samples and the placebo samples that underwent the freeze-thaw cycling. These results indicate that there was no degradation of gemfibrozil or excipients as a result of the freeze-thaw cycling.

### Example 12

The stability of gemfibrozil in five different liquid pharmaceutical compositions comprising gemfibrozil (15 mg/ml) in a carbonate buffer having a pH of 10.5 (+/−0.5), as well as the appearance, pH, density, and preservative efficacy of the liquid pharmaceutical compositions comprising gemfibrozil in the presence or absence of a preservative, were evaluated at 1 month under storage conditions of approximately 2-8° C., 25° C., and 40° C. with 60% relative humidity. Table 39 below describes the formulation composition of the liquid pharmaceutical compositions comprising gemfibrozil.

TABLE 39

| | Formulation Composition | | | | | | | | | |
|---|---|---|---|---|---|---|---|---|---|---|
| | AP | | BP | | CP | | DP | | EP | |
| | | Mass | | Mass | | Mass | | Mass | | Mass |
| Excipient* | % w/w | (g) | % w/w | (g) | % w/w | (g) | % w/w | (g) | % w/w | (g) |
| Buffer Portion | 89.2 | 89.2 | 89.2 | 89.2 | 89.2 | 89.2 | 89.2 | 89.2 | 89.2 | 89.2 |
| 1 | | | | | | | | | | |
| Gemfibrozil | | | | | | | | | | |
| Cherry Black | 0.0989 | 0.989 | 0.0989 | 0.989 | 0.0989 | 0.989 | 0.0989 | 0.989 | 0.0989 | 0.989 |
| Powder | | | | | | | | | | |
| Sodium | 0.0495 | 0.495 | 0.0495 | 0.495 | 0.0495 | 0.495 | 0.0495 | 0.495 | 0.0495 | 0.495 |
| saccharin | | | | | | | | | | |
| dihydrate | | | | | | | | | | |
| Benzalkonium | | | 0.015 | 0.150 | | | | | | |
| Chloride | | | | | | | | | | |
| Disodium | | | | | 0.200 | 2.00 | | | | |
| EDTA | | | | | | | | | | |
| Domiphen | | | | | | | 0.200 | 2.00 | | |
| Bromide | | | | | | | | | | |
| (BHA) | | | | | | | | | 0.100 | 1.00 |
| 10% HCl or | Adjust to pH 10.5 (+/−0.05) | | | | | | | | | |
| NaOH, 1M | | | | | | | | | | |
| solution (pH | | | | | | | | | | |
| adjustment) | | | | | | | | | | |
| Deionised | q.s. | q.s. | q.s. | q.s. | q.s. | q.s. | q.s. | q.s. | q.s. | q.s. |
| water to up | | | | | | | | | | |
| Total | 100 | 1000 g | 100 | 1000 g | 100 | 1000 g | 100 | 1000 g | 100 | 1000 g |

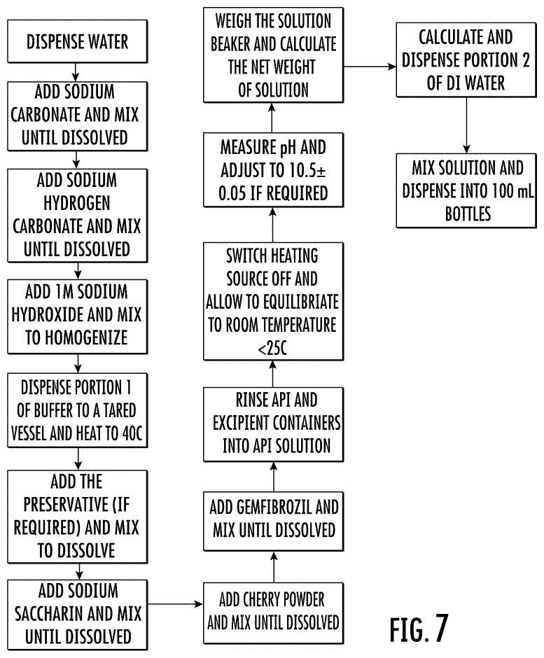

Five prototypes of the liquid pharmaceutical composition samples were prepared according to the method outlines in FIG. 7. Prototype AP was preservative free; Prototype BP included benzalkonium chloride; Prototype CP included disodium EDTA; Prototype DP included domiphen bromide; and Prototype EP included butyl hydroxyanisole (BHA).

During preparation of Prototype EP, comprising gemfibrozil and BHA, the solution turned dark red upon addition of BHA. Accordingly, Prototype EP was rejected according to the specifications and not progressed further. An attempt to replace BHA with butyl hydroxytoluene (BHT) at the same concentration with the addition of BHT at 60° C., the BHT did not dissolve and the prototype was not progressed further.

During preparation of Prototype BP, comprising gemfibrozil and benzalkonium chloride, crystals formed upon cooling down to ambient temperature after the addition of flavor and sweetener. Accordingly, Prototype BP was rejected according to the specifications and not progressed further.

Prototype AP, Prototype CP, and Prototype DP were progressed forward. Following preparation, the solutions were clear, colorless to pale yellow, and free from crystalline precipitate. The prototypes were stored for 1 month under storage conditions of approximately 2-8° C., 25° C., and 40° C. and evaluated. Table 40 below describes the appearance, pH, and whether or not related substances to gemfibrozil were formed.

TABLE 40

| | | | | | pH | Related Substances | | | | |
|---|---|---|---|---|---|---|---|---|---|---|
| | | Storage | | | (mean | RRT | RRT | RRT | RRT | |
| Formulation | Timepoint | Condition | | Appearance | of n = 2) | 0.814 | 0.808 | 0.858 | 0.878 | Total |
| POL033/012A | Initial | N/A | | Clear, | 10.51 | | | | | |
| No | | | | colourless | | | | | | |
| preservative | | | | solution, free | | | | | | |
| POL033/012C | | | | from any | 10.53 | | | | | |
| EDTA | | | | particulates | | | | | | |
| POL033/014D | | | | | 10.54 | | | | | |
| Domiphen | | | | | | | | | | |
| Bromide | | | | | | | | | | |
| POL033/012A | 1 month | 2-8° | C. | Clear, | 10.53 | | | | | |
| No | | | | colourless | | | | | | |
| preservative | | | | solution. | | | | | | |
| | | | | Large | | | | | | |
| | | | | crystals | | | | | | |
| | | | | sitting at the | | | | | | |
| | | | | bottom of | | | | | | |
| | | | | the solution. | | | | | | |
| | | | | After sitting | | | | | | |
| | | | | at room | | | | | | |
| | | | | temperature | | | | | | |
| | | | | for a few | | | | | | |
| | | | | hours all | | | | | | |
| | | | | crystals | | | | | | |
| | | | | dissolved | | | | | | |
| | | 25° | C. | Clear, | 10.50 | | | | | |
| | | | | colourless | | | | | | |
| | | | | solution, free | | | | | | |
| | | | | from any | | | | | | |
| | | | | particulates | | | | | | |
| | | 40° | C. | Clear, | 10.40 | | | | | |
| | | | | colourless | | | | | | |
| | | | | solution, free | | | | | | |
| | | | | from any | | | | | | |
| | | | | particulates | | | | | | |
| POL033012C | 1 month | 2-8° | C. | Clear, | 10.54 | | | | | |
| EDTA | | | | colourless | | | | | | |
| | | | | solution. | | | | | | |
| | | | | Large | | | | | | |
| | | | | crystals | | | | | | |
| | | | | sitting at the | | | | | | |
| | | | | bottom of | | | | | | |
| | | | | the solution. | | | | | | |
| | | | | After sitting | | | | | | |
| | | | | at room | | | | | | |
| | | | | temperature | | | | | | |
| | | | | for a few | | | | | | |
| | | | | hours all | | | | | | |
| | | | | crystals | | | | | | |
| | | | | dissolved | | | | | | |
| | | 25° | C. | Clear, | 10.51 | 0.06 | 0.15 | | | 0.21 |
| | | | | colourless | | | | | | |
| | | | | solution, free | | | | | | |
| | | | | from any | | | | | | |
| | | | | particulates | | | | | | |
| | | 40° | C. | Clear, | 10.42 | 0.25 | 0.11 | 0.20 | 0.20 | 0.76 |
| | | | | colourless | | | | | | |
| | | | | solution, free | | | | | | |
| | | | | from any | | | | | | |
| | | | | particulates | | | | | | |
| POL033014D | 1 month | 2-8° | C. | Clear, | 10.57 | | | | | |
| Domiphen | | | | colourless | | | | | | |
| Bromide | | | | solution, free | | | | | | |
| | | | | from any | | | | | | |
| | | | | particulates | | | | | | |
| | | 25° | C. | Clear, | 10.54 | | | | | |
| | | | | colourless | | | | | | |
| | | | | solution, free | | | | | | |
| | | | | from any | | | | | | |
| | | | | particulates | | | | | | |
| | | 40° | C. | Clear, | 10.43 | | | | | |
| | | | | colourless | | | | | | |
| | | | | solution, free | | | | | | |
| | | | | from any | | | | | | |
| | | | | particulates | | | | | | |

Prototype AP comprising gemfibrozil and free from preservative was clear, colorless, free from crystalline precipitate following storage for 1 month at 25° C. and 40° C. Large crystals were observed following storage for 1 month at 2-8° C., but these were dissolved following a few hours sitting at room temperature. The gemfibrozil assay demonstrated that the active gemfibrozil was within the 90.0-110.0 specification and generally close to target (100%). The formation of related substances following storage for 1 month at of 2-8° C., 25° C., and 40° C. did not occur.

Prototype DP comprising gemfibrozil and domiphen bromide was clear, colorless, and free from crystalline precipitate following storage for 1 month at 2-8° C., 25° C., and 40° C. The gemfibrozil assay demonstrated that the active gemfibrozil was within the 90.0%-110.0% specification and generally close to target (100%). At the initial point, domiphen bromide co-eluted with gemfibrozil influencing the initial peak to be 106%, but after 1-month of storage the gemfibrozil and domiphen bromide peaks were mostly separated with minimal impact on the gemfibrozil assay. The formation of related substances following storage for 1 month at of 2-8° C., 25° C., and 40° C. did not occur.

Prototype CP comprising gemfibrozil and EDTA was clear, colorless, and free from crystalline precipitate following storage for 1 month at 25° C. and 40° C. Large crystals were observed following storage for 1 month at 2-8° C., but these were dissolved following a few hours sitting at room temperature. The gemfibrozil assay demonstrated that the active gemfibrozil was within the 90.0-110.0 specification and generally close to target (100%). The formation of related substances following storage for 1 month at of 2-8° C. did not occur. Table 41 demonstrates that the formation of related substances following storage for 1 month occurred at of 25° C. and 40° C. The area of the peaks for storage at 25° C. were within specification limits (total impurities should have an increase of <1.00%, and any individual impurities should have an increase<0.20% of the correspondence peak area). However, the area of the peaks for storage at 40° C. exceeded specification limits in that three of the individual peaks were greater than or equal to 0.20%. Total peak area was still <1.0%. Regarding the individual peaks that exceeded specification limits, these changed over time. The increase in impurities at ˜6.1 to ˜6.7 minutes was accompanied by a decrease in purities (anticipated to be excipient related) at ˜1.5 minutes, which indicates that the degradation process may be excipient interactions rather than gemfibrozil degradation.

Preservative efficacy testing (PET) of Prototype AP demonstrated the self-preserving efficacy of the liquid pharmaceutical composition comprising gemfibrozil determined using USP 42 <51> test method. Prototype AP, as well as Prototype CP and Prototype DP were also assessed for preservative efficacy according to Ph. Eur. 10.2 (5.1.3) standards illustrated below in Table 41 at both 14 days and 28 days.

TABLE 41

| Analysis | Specification | Results |
|---|---|---|
| Preservative | Complies with Ph. Eur. 10/2 | Complies with Ph. |
| Efficacy Testing | (5.1.3) <br> | Eur. 10.2 (5.1.3) |
| | Bacteria: 3 at day 14; no | |
| | increase at 28 days (log | |
| | reduction) <br> | |
| | Yeast & moulds: 1 at day 14; | |
| | no increase at 28 days (log | |
| | reduction) | |

The preservative efficacy of Prototype AP comprising gemfibrozil complied with Ph. Eur. 10.2 (5.1.3) as shown below in Table 42. Prototype AP was effective at keeping organism growth at suitable levels although this was borderline for Aspergillus brasiliensis (A. brasiliensis). Notably, EDTA did not provide significant further preservative effect and had a lesser drop at 28 days as shown in Table 43.

TABLE 42
Sample - POL033/012C Preservative Free Solution

| | Time- | | | Specification |
|---|---|---|---|---|
| Organism | point | Results | Units | Ph Eur |
| A.brasiliensis | Inoculum | 5.45 | Log Reduction | Yeast & |
| | 14 days | 1.09 | Log Reduction | moulds: 1 |
| | 28 days | 1.47 | Log Reduction | log drop at |
| | | | | day 14; |
| | | | | no increase at |
| | | | | 28 days |
| C. albicans | Inoculum | 5.54 | Log Reduction | Yeast & |
| | 14 days | >4.54 | Log Reduction | moulds: 1 |
| | 28 days | >4.54 | Log Reduction | log drop at |
| | | | | day 14; |
| | | | | no increase at |
| | | | | 28 days |
| P. aeruginosa | Inoculum | 5.48 | Log Reduction | Bacteria: 3 log |
| | 14 days | >4.48 | Log Reduction | drop at day 14; |
| | 28 days | >4.48 | Log Reduction | no increase at |
| | | | | 28 days |
| E. coli | Inoculum | 5.62 | Log Reduction | Bacteria: 3 log |
| | 14 days | >4.62 | Log Reduction | drop at day 14; |
| | 28 days | >4.62 | Log Reduction | no increase at |
| | | | | 28 days |
| S. aureus | Inoculum | 5.46 | Log Reduction | Bacteria: 3 log |
| | 14 days | >4.46 | Log Reduction | drop at day 14; |
| | 28 days | >4.46 | Log Reduction | no increase at |
| | | | | 28 days |

The preservative efficacy of Prototype CP comprising gemfibrozil and EDTA complied with Ph. Eur. 10.2 (5.1.3) as shown below in Table 43. Prototype CP was effective at keeping organism growth at suitable levels although this was borderline for A. brasiliensis.

TABLE 43
Sample - POL033/012C Disodium EDTA

| | Time- | | | Specification |
|---|---|---|---|---|
| Organism | point | Results | Units | Ph Eur |
| A. brasiliensis | Inoculum | 5.45 | Log Reduction | Yeast & |
| | 14 days | 1.13 | Log Reduction | moulds: 1 |
| | 28 days | 1.30 | Log Reduction | log drop at |
| | | | | day 14; |
| | | | | no increase at |
| | | | | 28 days |
| C. albicans | Inoculum | 5.54 | Log Reduction | Yeast & |
| | 14 days | >4.54 | Log Reduction | moulds: 1 |
| | 28 days | >4.54 | Log Reduction | log drop at |
| | | | | day 14; |
| | | | | no increase at |
| | | | | 28 days |
| P. aeruginosa | Inoculum | 5.48 | Log Reduction | Bacteria: 3 log |
| | 14 days | >4.48 | Log Reduction | drop at day 14; |
| | 28 days | >4.48 | Log Reduction | no increase at |
| | | | | 28 days |
| E. coli | Inoculum | 5.62 | Log Reduction | Bacteria: 3 log |
| | 14 days | >4.62 | Log Reduction | drop at day 14; |
| | 28 days | >4.62 | Log Reduction | no increase at |
| | | | | 28 days |
| S. aureus | Inoculum | 5.46 | Log Reduction | Bacteria: 3 log |
| | 14 days | >4.46 | Log Reduction | drop at day 14; |
| | 28 days | >4.46 | Log Reduction | no increase at |
| | | | | 28 days |

The preservative efficacy of Prototype DP comprising gemfibrozil and domiphen bromide complied with Ph. Eur. 10.2 (5.1.3) as shown below in Table 44. Prototype DP was effective keeping organism growth at suitable levels and even decreasing growth.

TABLE 44
Sample - POL033/014D Domiphen Bromide

| | Time- | | | Specification |
|---|---|---|---|---|
| Organism | point | Results | Units | Ph Eur |
| A. brasiliensis | Inoculum | 5.45 | Log Reduction | Yeast & |
| | 14 days | 2.05 | Log Reduction | moulds: 1 |
| | 28 days | 4.27 | Log Reduction | log drop at |
| | | | | day 14; |
| | | | | no increase at |
| | | | | 28 days |
| C. albicans | Inoculum | 5.54 | Log Reduction | Yeast & |
| | 14 days | >4.54 | Log Reduction | moulds: 1 |
| | 28 days | >4.54 | Log Reduction | log drop at |
| | | | | day 14; |
| | | | | no increase at |
| | | | | 28 days |
| P. aeruginosa | Inoculum | 5.48 | Log Reduction | Bacteria: 3 log |
| | 14 days | >4.48 | Log Reduction | drop at day 14; |
| | 28 days | >4.48 | Log Reduction | no increase at |
| | | | | 28 days |
| E. coli | Inoculum | 5.62 | Log Reduction | Bacteria: 3 log |
| | 14 days | >4.62 | Log Reduction | drop at day 14; |
| | 28 days | >4.62 | Log Reduction | no increase at |
| | | | | 28 days |
| S. aureus | Inoculum | 5.46 | Log Reduction | Bacteria: 3 log |
| | 14 days | >4.46 | Log Reduction | drop at day 14; |
| | 28 days | >4.46 | Log Reduction | no increase at |
| | | | | 28 days |

Taste testing was performed on Prototype AP, Prototype CP, and Prototype DP. The taste of Prototype AP was acceptable. The taste of Prototype CP (EDTA) was ranked second with a slightly unacceptable overall taste and aftertaste. Prototype DP (domiphen bromide) was ranked third as very unpleasant having an overall bitter taste and after taste.

Liquid pharmaceutical compositions Prototype AP, Prototype CP, and Prototype DP were tested for pH. All samples demonstrated pH values within the set specifications (pH 10.5+/−0.5) as shown below in Table 45.

TABLE 45

| | | | Results at approximately 5 months | | | | | Specification |
|---|---|---|---|---|---|---|---|---|
| Organism | Timepoint | Initial | 2-8° C. | 25° C./60% RH | 30° C./65% RH | 40° C./75% RH | Units | Ph Eur |
| A. | Inoculum | 5.59 | 5.46 | 5.46 | 5.46 | 5.46 | Log | Yeast & |
| brasiliensis | 14 days | 0.53 | 2.01 | 0.80 | 0.15 | −0.02 | Log | moulds: 1 log |
| | | | | | | | Reduction | at 14 days; no |
| | 28 days | 0.44 | 2.36 | 0.65 | 0.36 | 0.16 | Log | increase at 28 |
| | | | | | | | Reduction | days |

| pH (USP) at closet | 10.5 | 10.5 | 10.4 | 10.3 | 10.3 | From DT-20-1511 v01 |
|---|---|---|---|---|---|---|
| timepoint | | | | | | |
| pH (Ph Eur) at | 10.5 | 10.5 | 10.4 | 10.4 | 10.3 | |
| closet timepoint | | | | | | |

While the present invention has been described in terms of its specific embodiments and specific examples, certain modifications and equivalents will be apparent to those skilled in the art and are intended to be included within the scope of the present invention.

1. A liquid pharmaceutical composition comprising:
gemfibrozil; and
an aqueous solvent; wherein
the aqueous solvent is a sodium carbonate/bicarbonate buffer having a pH of 10.5 (+/−0.5), and
the gemfibrozil is soluble in the pharmaceutical liquid composition at a final concentration of 5 mg/ml to 32 mg/ml.

2-3. (canceled)

4. The liquid pharmaceutical composition of claim 1, wherein the gemfibrozil is stable for up to 24 months at a temperature of 2-40° C., wherein during the 24 months of storage
there is no detectable increase in gemfibrozil-related substances,
the pH of the liquid pharmaceutical composition is 10.5 (+/−0.5), and
appearance of the liquid pharmaceutical composition does not change significantly such that the liquid pharmaceutical composition remains clear, is colorless or pale yellow, and is free from crystalline precipitate.

5-8.

9. The liquid pharmaceutical composition of claim 1, wherein preservative efficacy of the liquid pharmaceutical composition is maintained and microbial growth is prevented during 24 months in storage.

10. (canceled)

11. The liquid pharmaceutical composition of claim 1 further comprising a sweetener, preferably wherein the sweetener is at least one of sucralose or sodium saccharin, more preferably the sweetener is sodium saccharin present at a final concentration in the range of 0.02% to 0.2% weight/volume.

12-14. (canceled)

15. The liquid pharmaceutical composition of claim 1 further comprising a taste modifier, preferably the taste modifier is at least one of a flavoring, a liquid cherry flavor, or a powdered cherry flavor.

16-17. (canceled)

18. The liquid pharmaceutical composition of claim 1, wherein an ionic strength of the liquid pharmaceutical composition is in the range of 50 mM to 100 mM.

19. The liquid pharmaceutical composition of claim 1 further comprising a preservative, preferably wherein the preservative is at least one of ethylenediaminetetraacetic acid (EDTA) or domiphen bromide.

20. (canceled)

21. The liquid pharmaceutical composition of claim 1 further comprising at least one of a pharmaceutically acceptable carrier or a pharmaceutically acceptable excipient.

22. The liquid pharmaceutical composition of claim 1 for the treatment of a lysosomal storage disorder, wherein the lysosomal storage disorder is selected from the group consisting of Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, Galactosialidosis, Neuronal Ceroid Lipofuscinoses, and neurodegenerative disorders including Alzheimer's disease, Parkinson's disease, Dementia with Lewy Bodies, Multiple System Atrophies, Multiple Sclerosis, Huntington's disease, and Amyotrophic Lateral Sclerosis.

23. (canceled)

24. A method of preparing a liquid pharmaceutical composition having gemfibrozil, the method comprising:
adding gemfibrozil to a solvent; and
mixing the gemfibrozil and the solvent until at least the gemfibrozil dissolves, wherein
the gemfibrozil is present in the pharmaceutical liquid composition at a final concentration of 5 mg/ml to 32 mg/ml, and
the pH of the liquid pharmaceutical composition is 10.5 (+/−0.5).

25. The method of claim 24, wherein the solvent is a sodium carbonate/bicarbonate buffer having a pH of 10.5 (+/−0.5).

26. (canceled)

27. The method of claim 24, wherein the mixing of the gemfibrozil and solvent occurs at 40° C. at least until the gemfibrozil is dissolved.

28. The method of claim 24 further comprising adding at least one of a sweetener or a tasted modifier to the composition, preferably wherein the sweetener is sodium saccharin and the sodium saccharin is passed through a 400 UM mesh prior to adding the sweetener to the composition, and the taste modifier is at least one of a flavoring, a liquid cherry flavor, or a powdered cherry flavor.

29-31. (canceled)

32. The method of claim 24 wherein, prior to the adding gemfibrozil to the solvent the method further comprises stirring the gemfibrozil with water to form a slurry, wherein the stirring occurs at 40° C. and the stirring is preferably in the range of 230 rpm to 400 rpm.

33. (canceled)

34. The method of claim 24 further comprising adding a preservative.

35. The method of claim 24 comprising adjusting the pH of the liquid pharmaceutical composition to 10.5 (+/−0.5), bringing the liquid pharmaceutical composition to volume with water, and cooling the liquid pharmaceutical composition to ambient temperature overnight.

36-37. (canceled)

38. A method of treating a subject having a lysosomal storage disorder comprising administering the liquid pharmaceutical composition of claim 1 to a subject in need thereof, wherein the treating of a subject comprises any one of curing, slowing down, or halting the progression of the lysosomal storage disorder, preferably wherein the lysosomal storage disorder is selected from Tay-Sachs disease, Sandoff disease, Fabry disease, Krabbe disease, Niemann-Pick disease, Gaucher disease, Hunter Syndrome, Alpha-mannosidosis, Aspartylglucosaminuria, Cholesteryl ester storage disease, Chronic Hexosaminidase A Deficiency, Cystinosis, Danon disease, Farber disease, Fucosidosis, Galactosialidosis, Neuronal Ceroid Lipofuscinoses, and neurodegenerative disorders including Alzheimer's disease, Parkinson's disease, Dementia with Lewy Bodies, Multiple System Atrophies, Multiple Sclerosis, Huntington's disease, and Amyotrophic Lateral Sclerosis.

39. (canceled)

40. The method of claim 38, wherein the administering comprises a route of administration comprising one or more routes selected from the group consisting of oral, sublingual, sublabial, buccal, and transmucosal.

41. The method of claim 38, wherein a concentration of the gemfibrozil in the liquid pharmaceutical composition is in the range of 5 mg/mL to 32 mg/ml and the dose of gemfibrozil used to treat the subject is in the range of 600 mg to 1800 mg per day, preferably wherein the administering occurs at least once daily.

42. (canceled)

43. The method of claim 38 further comprising co-administering the pharmaceutical liquid composition with an anti-epilepsy drug, wherein the co-administering occurs at least once daily.

44-45. (canceled)
